(19) United States
(12) Patent Application Publication
(10) Pub. No.: US 2007/0107082 A1
(43) Pub. Date: May 10, 2007

(54) Transgenic monocotyledonous plants overexpressing a nhx protein and having improved growth characteristics and a method for making the same

(75) Inventors: Ana Isabel Sanz Molinero, Gentbrugge (BE); Valerie Frankard, Sint-Genesius-Rode (BE)

Correspondence Address: THE WEBB LAW FIRM, P.C., 700 KOPPERS BUILDING, 436 SEVENTH AVENUE, PITTSBURGH, PA, 15219, US

(73) Assignee: CropDesign N.V., Zwijnaarde (BE)

(21) Appl. No.: 10/559,097

(22) PCT Filed: Jun. 3, 2004
(86) PCT No.: PCT/EP04/51022

§ 371 Date: Sep. 19, 2006

(30) Foreign Application Priority Data
Jun. 3, 2003 (EP) 03076719.8

(51) Int. Cl. A01H 5/00; C12N 15/82

(52) U.S. Cl. 800/287; 800/320.1; 800/320.2

(57) ABSTRACT

The present invention concerns a method for improving plant growth characteristics, comprising increasing, in a monocotyledonous plant, expression of a nucleic acid encoding an NHX protein and/or increasing activity of an NHX protein, wherein said plant is grown under non-salt stress conditions.

The present invention relates generally to the field of molecular biology and concerns a method for improving the growth characteristics of monocotyledonous plants. More specifically, the present invention concerns a method for improving the growth characteristics of monocotyledonous plants by increasing, in a monocotyledonous plant, expression of a nucleic acid encoding an NHX protein and/or by increasing activity of an NHX protein. The present invention also concerns monocotyledonous plants having increased expression of a nucleic acid encoding an NHX protein and/or increased activity of an NHX protein, which plants have improved growth characteristics relative to corresponding wild type monocotyledonous plants. The plants are grown under non-salt stress conditions and have improved growth characteristics compared to their wild type counterparts also grown under corresponding conditions.

The ever-increasing world population and the dwindling supply of arable land available for agriculture fuel agricultural research towards improving the efficiency of agriculture and to increase the diversity of plants in horticulture. Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic components that may not always result in the desirable trait being passed on from parent plants. Advances in molecular biology have allowed mankind to manipulate the germplasm of animals and plants. Genetic engineering of plants entails the isolation and manipulation of genetic material (typically in the form of DNA or RNA) and the subsequent introduction of that genetic material into a plant. Such technology has led to the development of plants having various. Improved economic, agronomic or horticultural traits. A trait of particular economic interest is yield. Yield is normally defined as the measurable produce of economic value from a crop. This may be defined in terms of quantity and/or quality. Yield is directly dependent on several factors, for example, the number and size of the organs, plant architecture (for example, the number of branches), seed production and more. Root development, nutrient uptake and stress tolerance may also be important factors in determining yield. Yield may therefore be increased by optimizing one of the above mentioned factors.

The ability to improve various growth characteristics of a plant would have many applications in areas such as crop enhancement, plant breeding, in the production of ornamental plants, aboriculture, horticulture and forest. Improving growth characteristics, such as yield may also find use in the production of algae for use in bioreactors (for the biotechnological production of substances such as pharmaceuticals, antibodies, or vaccines, or for the bioconversion of organic waste) and other such areas.

It has now been found that the growth characteristics of a monocotyledonous plant may be improved by increasing expression in a monocotyledonous plant of a nucleic acid encoding an NHX protein and/or by increasing activity in a monocotyledonous plant of an NHX protein, which monocotyledonous plant is grown under non-salt stress conditions. The improved growth characteristics are improved as compared to the growth characteristics of wild type counterparts grown under corresponding conditions.

The NHX protein is a sodium antiporter and as an active sodium pump the NHX protein is involved in extruding Na+ ions from the cytoplasm into the vacuole of a cell. This is one of the mechanisms used by a plant to protect the cells against high salinity in the soil and in the water. NHX genes have been isolated from a number of plant species, such as Arabidopsis (Gaxiola et al. 1999. PNAS 96(4), 1480-1485), rice (OsNHX (Fukuda et al. Molecular cloning and expression of the Na+/H+ exchanger gene In Oryza sativa. Biochim Biophys Acta. 1999 Jul. 7; 1446(1-2):149-55) and from Atriplex (AgNHX, JP2000157287).

Transgenic plants overexpressing the Arabidopsis gene AtNHX have been shown to have increased tolerance to high salinity (200 to 400 mM NaCL) in growth media. Examples of salt-tolerant Arabidopsis plants, tomato plants and Brassica have been described. (Apse et al., Salt tolerance conferred by overexpression of a vacuolar Na+/H+ antiporter in Arabidopsis. Science. 1999 Aug. 20; 285 (5431): 1256-8; Apse MP, Blumwald E. Engineering salt tolerance in plants. Curr Opin Biotechnol. 2002 April; 13(2):146-50; Zhang and Blumwald Transgenic salt-tolerant tomato plants accumulate salt in foliage but not in fruit. Nat. Biotechnol. 2001 August; 19(8):7658; Zhang et al. Engineering salt-tolerant Brassica plants: characterization of yield and seed oil quality in transgenic plants with increased vacuolar sodium accumulation. Proc Natl Acad Sci USA 2001 Oct. 23; 98(22):12832-6). Transgenic Brassica napus plants overexpressing AfNHX were able to grow, flower, and produce seeds in the presence of 200 mM sodium chloride. Although transgenic plants grown in high salinity accumulated sodium to up to 6% of their dry weight, growth of the these plants was only marginally affected by the high salt concentration. Moreover, seed yield and the seed oil quality were not affected by the high salinity of the soil.

Furthermore, salt tolerant monocots were generated by transformation into plants of an NHX gene. Ohta et al. (FEBS Lett. 2002 Dee 18; 532(3):279-82) engineered a salt-sensitive rice cultivar (Oryza sativa. Kinuhikari) to express a vacuolar-type Na+/H+ antiporter gene from the halophytic plant, Atriplex gmelini (AgNHX). The activity of the vacuolar-type Na+/H+antiporter in the transgenic rice plants was eight-fold higher than that in wild-type rice plants. Salt tolerance assays followed by non-salt stress treatments showed that the transgenic plants overexpressing AgNHX could survive under conditions of 300 mM NaCl for 3 days whilst the wild-type rice plants could not. This indicates that overexpression of the Na+/H+ antiporter gene in rice plants significantly improves their salt tolerance. After salt-stress treatments, the surviving transgenic rice plants were transferred to soil conditions without salt stress and were grown in a greenhouse. Although the number of tillers was reduced compared to untreated transgenic rice plants, the transgenic rice plants grew until the flowering stage and set seeds after 3.5 months, demonstrating that the salt shock did not completely damage the fertility of the transgenic rice plants. All these transgenic plants showed better survival capacity when grown on high salinity media and showed “wild-type phenotypes” on the green biomass level and on the level of flowering and seed-production, whist the non-transgenic plants were suffering from salt toxicity. In tomato, the fruits of transgenic plants were smaller than the fruits of wild-type non-salt stressed plants. In summary, several reports have established a role for NHX genes in salt tolerance.

It was therefore unexpected to find that the growth characteristics of monocotyledonous plants could be improved by increasing expression in plants of a nucleic acid encoding an NHX protein and/or by increasing activity of an NHX protein, which monocotyledonous plants were grown under non-salt stress conditions.

Therefore according to the present invention, there is provided a method for improving the growth characteristics of monocotyledonous plants, comprising increasing, in a monocotyledonous plant, expression of a nucleic acid encoding an NHX protein and/or by increasing activity of an NHX protein, which monocotyledonous plant is grown under non-salt stress conditions. The growth characteristics are improved as compared to the growth characteristics of wild type counterparts grown under corresponding conditions.

The term “non-salt stress conditions” refers to sodium chloride levels in soil of below 15 mM. For some species even higher amounts of salt may be tolerated without any signs of stress.

A preferred method for increasing expression of a nucleic acid encoding an NHX protein and/or for increasing activity of an NHX protein comprises introducing into a (monocotyledonous) plant a nucleic acid encoding an NHX protein or a homologue, derivative or active fragment thereof. The nucleic acid may be introduced into a plant by, for example, plant transformation.

The NHX protein is a sodium antiporter. OsNHX proteins are Na+/H+ antiporters located at the vacuolar membranes and function in excluding Na+ from the cytosol to the vacuole in response to the electrochemical H+ gradient. A characteristic of mammalian Na+/H+ antiporters is their inhibition by amiloride. A putative amiloride binding site has been defined in HsNHX1: DVFFLFLLPPI. This motif is highly conserved in yeast and plant NHX genes (Gaxiola et al. 1999 PNAS; Yolio et al. Plant J. 2002 June; 30(5): 529-539). NHX proteins may therefore readily be identified by the presence of the following consensus sequence for the amioride (sodium) binding site: FFXXLLPPII, where X may be any amino acid.

NHX proteins may also readily be identified by carrying out assays to determine Na+/H+ activity. Such assays are well known in the art and include, for example, (i) functional complementation assays in yeast followed by determination of Na+/and art K+ content (Yokoi et al. 2002 (Plant J. Jun. 30(5): 529-539); Yamaguchi et al. 2003 (PNAS Vol. 100, No. 21, Oct. 14, 2003, pp 12510-12515); (ii) transport assays, as for example described by Yamaguchi et al. 2003; and (iii) transforming wild type yeast with the NHX gene of interest and cultivating under increasing salt concentrations and assessing the differential growth behaviour between wild type and transformed strains.

The expression “NHX protein” as used herein refers to a protein having: (i) the following consensus sequence: FFXXOLLPPII; and (ii) having (in increasing order of preference) at least 20%, 25%, 30%, 35%, 40%, 45%, 50%, 55%, 60%, 65%, 70%, 75%, 80%, 85%, 90%, 95% or more sequence identity to the sequence represented by SEQ ID NO: 2; and/or (iii) having Na+/H+activity. Any nucleic acid encoding a protein falling within the aforementioned definition may be suitable for use in the methods of the invention. NHX proteins falling under the aforementioned definition are referred to herein as “essentially similar” to the sequence represented by SEQ ID NO 2. A gene encoding an NHX protein is a gene essentially similar to the sequence represented by SEQ ID NO 1. The term “essentially similar” to SEQ ID NO 1 or SEQ ID NO: 2 includes SEQ ID NO 1 or SEQ ID NO 2 itself and includes homologues, derivatives and active fragments of SEQ ID NO: 2 and includes portions of SEQ ID NO: 1 and sequences capable of hybridising to the sequence of SEQ ID NO: 1. The sequence of SEQ ID NO 1 has previously been deposited in the GenBank under the accession number AB021878 and the corresponding protein, SEQ ID NO 2, has been deposited in GenBank under accession number BAA83337.

The term “essentially similar to” also includes a complement of the sequences of SEQ ID NO: 1 or SEQ ID NO: 2; RNA, DNA, a cDNA or a genomic DNA corresponding to the sequences of SEQ ID NO: 1 or SEQ ID NO: 2; a variant of the gene or protein due to the degeneracy of the genetic code; allelic variant of the gene or protein; and different splice variants of the gene or protein and variants that are interrupted by one or more intervening sequences. The term “essentially similar to” also includes a family member or homologues, orthologues and paralogues of the gene or protein represented by the sequence of SEQ ID NO: 1 or SEQ ID NO: 2. Moreover, the conservation of NHX genes among diverse prokaryotic and eukaryotic species also allows the use of non-plant NHX genes for the methods of the present invention, such as NHX genes/proteins from yeast, fungi, moulds, algae, plants, insects, animals, human etc.

It should be clear that the applicability of the invention is not limited to use of a nucleic acid represented by SEQ ID NO 1 nor to the nucleic acid sequence encoding an amino acid sequence of SEQ ID NO 2, but that other nucleic acid sequences encoding homologues, derivatives or active fragments of SEQ ID NO 2 may be useful in the methods of the present invention. Nucleic acids suitable for use in the methods of the invention include those encoding NHX proteins according to the aforementioned definition, i.e. having: (i) the following consensus sequence: FFXXLLPPII; and (ii) having (in increasing order of preference) at least 20%, 25%, 30%, 35%, 40%, 45%, 50%, 55%, 60% Y, 65%, 70%, 75%, 80%, 85%, 90%, 95% or more sequence identity to the sequence represented by SEQ ID NO: 2; and/or (iii) having Na+/H+ activity.

Examples of NHX proteins failing within the aforementioned definition include:

AtNHX1 (AF106324: SEQ ID NO: 4 encoded by the sequence of SEQ ID NO: 3), AtNHX2 (AG009465), AtNHX3 (AC011623), AtNHX4 (AB015479), AtNHX5 (AC005287) and AtNHX6 (AC010793) all from Arabidopsis and described in Yokoi et al. 2002. AF007271 represents a cDNA encoding the AtNHX1 protein, the other accession numbers correspond to the BAC number in which genomic sequence for AtNHX 2 to 6 is given. Prediction programs, which are well known in the art, may be used to identify the coding region of genes.

Table 1 below (which is taken from Yokoi et al., 2002) shows the sequence identities between the different Arabidopsis NHX proteins. Sequence identity was calculated as described in Yokoi et al., 2002.

TABLE 1
Amino acid similarity comparison of the six Arabidopsis thaliana
family members of AtNHX Na+/H+ antiporters

| | AtNHX1 | AtNHX2 | AtNHX3 | AtNHX4 | AtNHX5 | AtNHX6 |
|---|---|---|---|---|---|---|
| | (AF106342) | (AC009465) | (AC011623) | (AB015479) | (AC005287) | [symbol] C010793) |
| AtNHX1 | — | 87.5% | 68.6% | 56.0% | 23.4% | 22.9% |
| (AF106342) | | | | | | |
| AtNHX2 | | — | 68.9% | 56.1% | 23.2% | 22.1% |
| (AC009465) | | | | | | |
| AtNHX3 | | | — | 55.4% | 23.4% | 21.7% |
| (AC011623) | | | | | | |
| AtNHX4 | | | | — | 23.8% | 21.9% |
| (AB015479) | | | | | | |
| AtNHX5 | | | | | — | 78.7% |
| (AC005287) | | | | | | |
| AtNHX6 | | | | | | — |
| (AC01O793) | | | | | | |

The family members are listed in order of sequence similarity beginning with the prototype AtNHX1. Accession numbers corresponding to the Arabidopsis genome sequencing project database are given in parentheses

Further examples of NHX proteins falling within the aforementioned definition include: SEQ ID NO: 5 and SEQ ID NO: 6 (AAR19085 from Medicago sativa); SEQ ID NO: 7 and SEQ ID NO: 8 (AAK53432 from Suaeda maritime), which are both dicots.

Examples of genes originating from monocotyledonous plants include: SEQ ID NO: 9 and SEQ ID NO: 10 from maize (AAP20428); SEQ ID NO: 11 and SEQ ID NO: 12 also from maize (AAP20429); SEQ ID NO: 13 and SEQ ID NO: 14 also from maize (AAP20430); SEQ ID NO: 15 and SEQ ID NO: 16 also from maize (AAP20431); SEQ ID NO: 17 and SEQ ID NO: 18 from barley (BAC56698); SEQ ID NO: 19 and SEQ ID NO: 20 from wheat (AAK76738); and SEQ ID NO: 21 and SEQ ID NO: 22 from rice (AAQ63678).

Examples of non-plant NHX proteins and their encoding genes, which also fall within the aforementioned definition include: SEQ ID NO: 23 and SEQ ID NO: 24 (AAB64861) from Saccharomyces cerevisiae; and SEQ ID NO: 25 and SEQ ID NO: 26 (EAA56466) from Magnaporthe grisea.

Other homologues include homologues from Hordeum vulgare (AB089197); Triticum aestivum (AY04246), Zea Mays (AF307944) and from Arabidopsis and Atriplex, as mentioned above.

Preferably, the nucleic acid encoding an NHX protein is introduced into a monocotyledonous plant in a sense direction coupled to a tissue-specific promoter or to a weak constitutive promoter.

The nucleic acid to be used in the methods of the present invention may be artificial, wild type or native or endogenous. Alternatively, the nucleic acid may be derived from another species, which gene is introduced into a plant as a transgene, for example by transformation. The nucleic acid may thus be derived (either directly or indirectly (if subsequently modified)) from any source. The nucleic acid may be isolated from a microbial source, such as bacteria, yeast or fungi, or from a plant, algae or insect or animal including human) source. This nucleic acid may be substantially modified from its native form in composition and/or genomic environment through deliberate human manipulation. The nucleic acid is preferably a homologous nucleic acid, i.e. a nucleic acid obtained from another plant, whether from the same plant species or different. The nucleic acid may be isolated from a monocotyledonous species, preferably from the family Poaceae, further preferably from the genus Oryza. More preferably, the nucleic acid is as represented by SEQ ID NO: 1 or a portion thereof or a nucleic acid sequence capable of hybridising therewith or is a nuclei acid encoding an amino acid represented by SEQ ID NO: 2 or a homologue, derivative or active fragment thereof.

According to the invention, enhanced or increased expression of an NHX protein-encoding nucleic acid is envisaged. Methods for obtaining enhanced or increased expression of genes or gene products are well documented in the art and include, for example, overexpression driven by an appropriate promoter, the use of transcription enhancers or translation enhancers. The term overexpression as used herein means any form of expression that is additional to the original wild-type expression level.

Alternatively or additionally, increased expression of an NHX-encoding gene and/or increased activity of an NHX protein in a plant cell may be achieved by mutagenesis of the plant cell. For example, the mutations may be responsible for altered control of the NHX encoded gene, resulting in more expression of the gene. The mutations may cause conformational changes of the protein resulting in more activity of the protein.

Mutations in the NHX gene may occur naturally and may form the basis of the selection of plants showing higher yield. These allelic variants may have improved activity, for example, via an improved affinity for ions. Therefore, according to another aspect of the invention, there is provided a method for the selection of pants having improved growth characteristics, which improved growth characteristics are based on increased activity of an NHX gene.

Methods for the search and identification of homologues of an NHX protein would be well within the realm of a person skilled in the art. The search and identification of homologous genes involves the screening of sequence information available, for example, in public databases, that include but are not limited to the DNA Database of Japan (DDBJ) http://www.ddbi.nig.ac.jpD; Genbank-(http://www.ncbi.nlm.nih.gov/web/Genbank/Index.html); and the European Molecular Biology Laboratory Nucleic Acid Sequence Database (EMBL) (http:/w.ebi.ac.uk/ebi-docs/embl-db.html) or versions thereof or the MIPS database. A number of different search algorithms have been developed, including but not limited to the suite of programs referred to as BLAST programs. Them am five implementations of BLAST, three designed for nucleotide sequence queries (BLASTN, BLASTX, and TBLASTX) and two designed for protein sequence queries (BLASTP and TBLASTN) (Coulson, Trends in Biotechnology: 76-80, 1994; Birren et al., GenomeAnalysis, 1:543, 1997). Such methods involve alignment and comparison of sequences. The BLAST algorithm calculates percent sequence identity and performs a statistical analysis of the similarity between the two sequences. The software for performing BLAST analysis is publicly available through the National Centre for Biotechnology Information. Other such software or algorithms are GAP, BESTFIT, FASTA and TFASTK. GAP uses the algorithm of Needleman and Wunsch (J. Mol. Biol. 48: 443-453, 1970) to find the alignment of two complete sequences that maximizes the number of matches and minimizes the number of gaps.

The homologous genes may belong to the same gene family as the gene corresponding to SEQ ID NO 1. The analysis of a gene family may be carried out using sequence similarity analysis. To perform this analysis one may use standard programs for multiple alignments e.g. Clustal W. A neighbour-joining tree of the proteins homologous to NHX may be used to provide an overview of structural and ancestral relationships. Sequence identity may be calculated using an alignment program as described above, for example with the program align X as a module of the VNTi suite 5,5 software package, using the standard parameters. In the Arabidopsis genome a number of family members of the NHX protein were identified (NHX1, NHX2, NHX3, NHX4 and NHX5, Yokoi et al. 2002 The plant journal 30, 529-539). Therefore it is expected that in other plants such as rice other family members of the NHX protein will later be identified. Advantageously, these family members may be useful in the methods of the invention.

The above-mentioned analyses for sequence homology is preferably carried out on a full-length sequence, but may also be based on a comparison of certain regions such as conserved domains. The identification of such domains, would also be well within the realm of the person skilled in the art and would involve, for example, a computer readable format of the nucleic adds of the present invention, the use of alignment software programs and the use of publicly available information on protein domains, conserved motifs and boxes. This information is available in the PRODOM (http://www.biochem.ucl.ac.uk/bsm/dbbrowser/jj/prodomsrchjj.html), PIR (http://pir.georgetown.edu/) or pFAM (http://pfam.wustl.edu/) database. Sequence analysis programs designed for motif searching may be used for identification of fragments, regions and conserved domains as mentioned above. Preferred computer programs include, but are not limited to, MEME, SIGNALSCAN, and GENESCAN. A MEME algorithm (Version 22) may be found in version 10.0 of the GCG package; or on the Internet site http://www.sdsc.ed/MEME/meme. SIGNALSCAN version 4.0 information is available on the Internet site http://biosci.cbs.umn.edu/software/sigscan.html. GENESCAN may be found on the Internet site http://www.gnomic.stanford.edu/GENESCANW.html.

A person skilled in the art may use the homologous sequences provided herein to find similar sequences in other species and other organisms. Sequences essentially similar to any of SEQ ID NO: 1 to SEQ ID NO: 26 may be recorded on a computer readable media. As used herein “computer readable media” refers to any medium that can be read and accessed directly by a computer. Such media include, but are not limited to magnetic storage media, such as floppy disc, hard disc, storage medium and magnetic tape, optical storage media such as CD-ROM, electrical storage media such as RAM or ROM. These readable formats will allow a skilled person to find homologues of the sequences in other plant species or other organisms.

“Homologues” of a protein encompass, peptides, oligopeptides, polypeptides, proteins and enzymes having amino acid substitutions, deletions and/or insertions relative to the unmodified protein in question and having similar biological and functional activity as the unmodified protein from which they are derived. To produce such homologues, amino acids of the protein may be replaced by other amino acids having similar properties (such as similar hydrophobicity, hydrophilicity, antigenicity, propensity to form or break α-helical structures or β-sheet structures). Conservative substitution tables are well known in the art (see for example Creighton (1984) Proteins. W. H. Freeman and Company). “Homologues” of a nucleic add encompass nucleic acids having nucleotide substitutions, deletions and/or insertions relative to the unmodified nucleic add in question and having similar biological and functional activity as the unmodified nucleic acid from which they are derived.

Suitable homologues are those having: (i) the following consensus sequence: FFXXLLPPII; and (ii) having (in increasing order of preference) at least 20%, 25%, 30%, 35%, 40%, 45%, 50%, 55%, 60%, 65%, 70%, 75%, 80%, 85%, 90%, 95% or more sequence identity to the sequence represented by SEQ ID NO: 2; and/or (iii) having Na+/H+ activity. Nucleic adds encoding such homologues are useful in the methods of the invention.

The percentage identity may be calculated using an alignment program as mentioned above. For example, the percentage identity may be calculated using the program GAP or the program align X, as a module of the vector NTI suite 5.5 software package, using standard parameters (for example GAP penalty 5, GAP opening penalty 15, GAP extension penalty 6.6).

Two special forms of homology, orthologous and paralogous, are evolutionary concepts used to describe ancestral relationships of genes/proteins. The term “paralogous” relates to gene-duplications within the genome of a species leading to paralogous genes. The term “orthologous” relates to homologous genes in different organisms due to ancestral relationship. The term “homologues” as used herein also encompasses paralogues and orthologues of the proteins useful in the methods according to the invention.

Othologues in, for example, monocot plant species may easily be found by performing a so-called reciprocal blast search. This may be done by a first blast involving blasting the sequence in question (for example, SEQ ID NO: 1 or SEQ ID NO: 2) against any sequence database, such as the publicly available NCBI database which may be found at: http://www.ncbi.nim.nih.gov. If orthologues in rice were sought, the sequence in question would be blasted against, for example, the 28,469 full-length cDNA clones from Olyza sativa Nipponbare available at NCBI. BLASTn or tBLASTX may be used when starting from nucleotides or BLASTP or TBLASTN when starting from the protein, with standard default values. The blast results may be filtered. The full-length sequences of either the filtered results or the non-filtered results are then blasted back (second blast) against the sequences of the organism from which the sequence in question is derived. The results of the first and second blasts are then compared. An orthologue is found when the results of the second blast give an NHX nucleic add or NHX polypeptide as hits with the highest similarity. If one of the organisms is rice, then a paralogue is found. In the case of large families, ClustalW may be used, followed by a neighbour joining tree, to help visualize the clustering.

“Substitutional variants” of an NHX protein are those in which at least one residue in an amino acid sequence has been removed and a different residue inserted in its place. Amino acid substitutions are typically of single residues, but may be clustered depending upon functional constraints placed upon the polypeptide; insertions will usually be of the order of about 1-10 amino acid residues, and deletions will range from about 1-20 residues. Preferably, amino acid substitutions comprise conservative amino add substitutions.

“Insertional variants” of an NHX protein are those in which one or more amino add residues are introduced into a predetermined site in a protein. Insertions may comprise amino-terminal and/or carboxy-terminal fusions as well as intra-sequence insertions of single or multiple amino adds. Generally, insertions within the amino acid sequence will be smaller than amino- or carboxy-terminal fusions, of the order of about 1 to 10 residues. Examples of amino- or carboxy-terminal fusion proteins or peptides include the binding domain or activation domain of a transcriptional activator as used in the yeast two-hybrid system, phage coat proteins, (histidine)₈tag, glutathione S-transferase-tag, protein A, maltose-binding protein, dihydrofolate reductase, Tag♦100 epitope, c-myc epitope, FLAG [symbol] -epitope, lacZ, CMP (calmodulin-binding peptide), HA epitope, protein C epitope and VSV epitope.

“Deletion variants” of an NHX protein are characterised by the removal of one or more amino adds from the protein. Amino add variants of a protein may readily be made using peptide synthetic techniques well known in the art, such as solid phase peptide synthesis and the like, or by recombinant DNA manipulations. The manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols.

The term “derivatves” of an NHX protein refers to peptides, oligopeptides, polypeptides, proteins and enzymes which may comprise substitutions, or deletions or additions of naturally and non-naturally occurring amino add residues compared to the amino acid sequence of a naturally-occurring form of the protein as presented in SEQ ID NO 2. “Derivatives” of a protein as presented in SEQ ID NO 2 encompass peptides, oligopeptides, polypeptides, proteins and enzymes which may comprise naturally occurring altered, glycosylated, acylated or non-naturally occurring amino acid residues compared to the amino acid sequence of a naturally occurring form of the polypeptide. A derivative may also comprise one or more non-amino acid substituents compared to the amino acid sequence from which it is derived, for example, a reporter molecule or other ligand, covalently or non-covalently bound to the amino acid sequence, such as a reporter molecule which is bound to facilitate its detection, and non-naturally occurring amino acid residues relative to the amino add sequence of a naturally-occurring protein.

“Active fragments” of an NHX protein encompasses at least five contiguous amino add residues of a protein, which residues retain similar biological and/or functional activity to the naturally occurring protein. A preferred fragment of an NHX protein is a C-terminal truncated version of the NHX protein, lacking one or more or all of the 100 last amino acids. Other preferred fragments are fragments of the NHX protein starting at the second or third or further internal methionin residues. A further preferred fragment comprises the motif: FFXXLLPPII, where X can be any amino add and/or wherein the protein has Na+/H+ activity.

Advantageously, the method according to the present invention may also be practised using portions (fragments) of DNA or of a nucleic acid sequence. The term “DNA fragment or DNA segment or portion” refers to a piece of DNA derived or prepared from an original (larger) DNA molecule, which DNA fragment or segment, when expressed in a plant, gives rise to plants having modified growth characteristics. The DNA fragment or segment may comprise many genes, with or without additional control elements, or may contain just spacer sequences etc. The fragment is preferably greater than 66 nucleotides and further preferably encodes a protein comprising the motif: FFXXLLPPII, where X can be any amino acid and/or wherein the protein has Na+/H+ activity. In the case where portions are to be used as molecular markers, or in other breeding applications, the portion may of course be much shorter.

An NHX gene suitable for the methods of the present invention also encompasses a nucleic add capable of hybridising with SEQ ID NO 1. The term “hybridisation” as defined herein is the process wherein substantially homologous complementary nucleotide sequences anneal to each other. The hybridisation process may occur entirely in solution, i.e. both complementary nucleic acids are in solution. Tools in molecular biology relying on such a process include the polymerase chain reaction (PCR; and all methods based thereon), subtractive hybridisation, random primer extension, nuclease S1 mapping, primer extension, reverse transcription, cDNA synthesis, differential display of RNAs, and DNA sequence determination. The hybridisation process may also occur with one of the complementary nucleic acids immobilised to a matrix such as magnetic beads, Sepharose beads or any other resin. Tools in molecular biology relying on such a process include the isolation of poly (A+) mRNA. The hybridisation process may furthermore occur with one of the complementary nucleic acids immobilised to a solid support such as a nitro cellulose or nylon membrane or immobilised by e.g. photolithography to e.g. a siliceous glass support (the latter known as nucleic add arrays or microarrays or as nucleic acid chips). Tools in molecular biology relying on such a process include RNA and DNA gel blot analysis, colony hybridisation, plaque hybridisation, in situ hybridisation and microarray hybridisation. In order to allow hybridisation to occur, the nucleic acid molecules are generally thermally or chemically denatured to melt a double strand into two single strands and/or to remove hairpins or other secondary structures from single stranded nucleic adds. The stringency of hybridisation is influenced by conditions such as temperature, salt concentration and hybridisation buffer composition. High stringency conditions for hybridisation include high temperature and/or low salt concentration (salts include NaCl and Na₃-citrate) and/or the inclusion of formamide in the hybridisation buffer and/or lowering the concentration of compounds such as SDS (detergent) in the hybridisation buffer and/or exclusion of compounds such as dextran sulphate or polyethylene glycol (promoting molecular crowding) from the hybridisation buffer. Conventional hybridisation conditions are described in, for example, Sambrook (2001) Molecular Cloning: a laboratory manual, 3rd Edition Cold Spring Harbor Laboratory Press, CSH, New York, but the skilled craftsman will appreciate that numerous different hybridisation conditions may be designed in function of the known or the expected homology and/or length of the nucleic add sequence. With specifically hybridising is meant hybridising under stringent conditions. Specific conditions for “specifically hybridising” are for example: hybridising under stringent conditions such as a temperature of 60° C. followed by washes in 2×SSC, 0.1×SDS, and 1×SSC, 0.1×SDS. Sufficiently low stringency hybridisation conditions are particularly preferred to isolate nucleic adds heterologous to the DNA sequences of the invention defined supra. Elements contributing to heterology include allelism, degeneration of the genetic code and differences in preferred codon usage.

The methods according to the present invention may also be practiced using an alternative splice variant of a nucleic add sequence encoding an NHX protein, for example a splice variant of SEQ ID NO 1. The term “alternative splice variant” as used herein encompasses variants of a nucleic add sequence in which introns and/or exons have been excised and/or replaced and/or added. Such variants will be ones in which the biological activity of the protein remains unaffected, which may be achieved by selectively retaining functional segments of the protein. Such splice variants may be found in nature or may be manmade. Methods for making such splice variants are well known in the art. Therefore according to another aspect of the present invention, there is provided, a method for improving the growth characteristics of monocotyledonous plants, comprising increasing expression in a plant of a nucleic add sequence encoding an alternative splice variant of an NHX protein and/or by modulating activity of a protein encoded by a splice variant of an NHX protein. The splice variant preferably encodes a protein comprising the motif FFXXLLPPII, where X can be any amino acid and/or Na+/H+ activity.

Another method for modifying plant growth characteristics resides in the use of allelic variants of a gone essentially similar to SEQ ID NO 1. Allelic variants exist in nature and encompassed within the methods of the present invention is the use of these natural alleles. “Allelic variants” are defined herein comprise single nucleotide polymorphisms (SNPs) as well as small insertion/deletion polymorphisms (INDELs; the size of INDELs is usually less than 100 bp). SNPs and INDELs form the largest set of sequence variants in naturally occurring polymorphic strains of most organisms. Differences between alleles are naturally occurring differences between the genes of different plants of the same species. These differences may be substitution and/or addition and/or deletion of for example 1, 2, 3 or more base pairs. The allelic variant preferably encodes a protein comprising the motif; FFXXLLPII, where X can be any amino acid and/or Na+/H+ activity.

Allelic variants may also find use in conventional breeding programs, such as in marker assisted breeding where it is sometimes practical to introduce allelic variation in the plants by mutagenic treatment of a plant. One suitable mutagenic method is EMS mutagenesis. Identification of allelic variants then takes place by, for example, PCRP. This is followed by a selection step for selection of superior allelic variants of the sequence in question and which give rise to altered growth characteristics. Selection is typically carried out by monitoring growth performance of plants containing different allelic variants of the sequence in question (for example SEQ ID NO 1). Monitoring growth performance may be done in a greenhouse or in the field. Further optional steps include crossing plants in which the superior allelic variant was identified with another plant. This could be used, for example, to make a combination of interesting phenotypic features.

According to another aspect of the present invention, advantage may be taken of the HNX nucleotide sequence in breeding programs. The nucleic acid sequence may be on a chromosome or on a part thereof. The breeding programs may be conventional breeding programs or marker-assisted breeding programs in which a DNA marker is identified which may be genetically linked to the HNX gene. This DNA marker is then used in breeding programs to select plants having improved growth characteristics.

The methods according to the present invention may also be practised by introducing into a plant at least a part of a (natural or artificial) chromosome (such as a Bacterial Artificial Chromosome (BAC)), which chromosome contains at least a gene/nucleic acid sequence encoding an NHX protein (such as for example a protein represented by SEQ ID NO 2), preferably together with one or more related gene family members. Therefore, according to a further aspect of the present invention, there is provided a method for improving the growth characteristics of plants by introducing into a plant at least a part of a chromosome comprising at last a gene/nucleic acid encoding an NHX protein.

The invention also provides genetic constructs and vectors to facilitate the introduction and/or expression and/or maintenance of the nucleic acid encoding an NHX protein and promoter into a plant cell, tissue or organ are provided. The constructs are suitable for use in the methods of the invention. Therefore, according to a further aspect of the present invention, there is provided a genetic construct comprising:
(i) a nucleic acid encoding an NHX protein;
(ii) a control sequence capable of regulating expression of the nucleic acid sequence of (i); and optionally
(iii) a transcription termination sequence.

The genetic construct may be an expression vector, wherein said nucleic add sequence is operably linked to one or more control sequences allowing expression in prokaryotic and/or eukaryotic host cells.

Preferably, the control sequence is a tissue-specific promoter, further preferably a seed-specific promoter, more preferably an endosperm-specific promoter, most preferably a prolamin promoter, such as for example a rice prolamin promoter (SEQ ID NO: 27).

The term “tissue-specific” promoter as defined herein refers to a promoter that is expressed predominantly in at least one tissue or organ. Preferably the tissue-specific promoter is a prolamin promoter, with predominant expression in the endosperm, or a promoter of similar strength and/or a similar expression pattern. Similar strength and/or similar expression pattern may be analysed for example by coupling the promoters to a reporter gene and checking the function of the reporter gene in tissues of the plant. One well known reporter gene is beta-glucuronidase and the colorimetric GUS staining which may be used to visualize reporter gene activity in plant tissues. Examples of other seed-specific promoters are shown in Table 2 below.

TABLE 2
Examples of seed-specific promoters suitable for
use in the performance of the present invention

| Gene | Expression | |
|---|---|---|
| source | pattern | Reference |
| seed-specific | seed | Simon, et al., Plant Mol. |
| genes | | Biol. 5: 191, 1985; Scofield, |
| | | et al., J. Biol. Chem. 262: |
| | | 12202, 1987.; Baszczynski, |
| | | et al., Plant Mol. Biol. |
| | | 14: 633, 1990. |
| Brazil Nut | seed | Pearson, et al., Plant Mol. |
| albumin | | Biol. 18: 235-245, 1992. |
| legumin | seed | Ellis, et al., Plant Mol. |
| | | Biol. 10: 203-214, 1988. |
| glutelin | seed | Takaiwa, et al., Mol. Gen. |
| (rice) | | Genet. 208: 15-22, 1986; |
| | | Takaiwa, et al., FEBS Letts. |
| | | 221: 43-47, 1987. |
| zein | seed | Matzke et al Plant Mol Biol, |
| | | 14(3): 323-32 1990 |
| napA | seed | Stalberg, et al., Planta |
| | | 199: 515-519, 1996. |
| wheat LMW | endosperm | Mol Gen Genet 216: 81-90, |
| and HMW | | 1989; NAR 17: 461-2, 1989 |
| gluterin-1 | | |
| wheat SPA | seed | Albani et al, Plant Cell, |
| | | 9: 171-184, 1997 |
| wheat α, | endosperm | EMBO 3: 1409-15, 1984 |
| β, γ- | | |
| gliadins | | |
| barley ltr1 | endosperm | |
| promoter | | |
| barley B1, | endosperm | Theor Appl Gen 98: 1253-62, |
| C, D, | | 1999; Plant J 4: 343-55, |
| hordein | | 1993; Mol Gen Genet 250: |
| | | 750-60, 1996 |
| barley DOF | endosperm | Mena et al, The Plant Journal, |
| | | 116(1): 53-62, 1998 |
| biz2 | endosperm | EP99106056.7 |
| synthetic | endosperm | Vicente-Carbajosa et al., |
| promoter | | Plant J. 13: 629-640, 1998. |
| rice prolamin | endosperm | Wu et al, Plant Cell |
| NRP33 | | Physiology 39(8) 885-889, |
| | | 1998 |
| rice -globulin | endosperm | Wu et al, Plant Cell |
| Glb-1 | | Physiology 39(8) 885-889, |
| | | 1998 |
| rice OSH1 | embryo | Sato et al, Proc. Natl. |
| | | Acad. Sci. USA, 93: |
| | | 8117-8122, 1996 |
| rice -globulin | endosperm | Nakase et al. Plant Mol. |
| REB/OHP-1 | | Biol. 33: 513-522, 1997 |
| rice ADP- | endosperm | Trans Res 6: 157-68, |
| glucose PP | | 1997 |
| maize ESR | endosperm | Plant J 12: 235-46, |
| gene family | | 1997 |
| sorgum | endosperm | PMB 32: 1029-35, |
| -kafirin | | 1996 |
| KNOX | embryo | Postma-Haarsma et al, |
| | | Plant Mol. Biol. 39: |
| | | 257-71, 1999 |
| rice oleosin | embryo and | Wu et al, J. Biochem., |
| | aleuron | 123: 386, 1998 |
| sunflower | seed (embryo | Cummins, et al., |
| oleosin | and dry seed) | Plant Mol. Biol. 19: |
| | | 873-876, 1992 |
| PRO0117, | weak in | |
| putative rice | endosperm | |
| 40S ribosomal | | |
| protein | | |
| PRO0135, | strong in | |
| rice alpha- | endosperm | |
| globulin | | |
| PRO0136, | weak in | |
| alanine amino- | endosperm | |
| transferase | | |
| PRO0147, | weak in | |
| trypsin | endosperm | |
| inhibitor | | |
| ITR1 (barley) | | |
| PRO0151, | embryo + | |
| rice WSI18 | stress | |
| PRO0175, | embryo + | |
| rice RAB21 | stress | |
| PRO0218, | aleurone + | |
| rice oleosin | embryo | |
| 18 kd | | |

Also preferred is the use of a weak constitutive promoter, such as Pros (the promoter of the nopaline synthase (nos) gene from A. tumefaciens), which is a well-characterised and widely used weak constitutive promoter with expression around 10 lower than detected for the p35S, in plants. Other weak constitutive promoters include: pAdh1-intron: the promoter of the maize alcohol dehydydrogenase I gene (without the pAdh1 intron 1 the promoter is a weak promoter with expression about 10× lower than the pAdh1 plus Intron 1 downstream of the Adh1 promoter). The Adh1 gene is regulated by anaerobiosis (inducible). A preferred weak constitutive promoter is the maize ubiquitin promoter having a deleted first intron (SEQ ID NO: 28).

Persons skilled in the art are well aware of the meaning of such terms as “strong constitutive promoters” and “weak constitutive promoters”. Nevertheless, the term “weak constitutive promoter” as defined herein refers to a promoter having expression levels that are 10× or less than a strong constitutive promoter. For example, an ubiquitin promoter (with its first intron) is generally known to be a strong constitutive promoter, and it is also known that deletion of the first intron reduces expression of the promoter ten fold rendering it a weak constructive promoter.

Therefore a preferred genetic construct for use in the methods of the invention comprises a tissue-specific promoter, preferably a seed-specific promoter, most preferably an endosperm-specific promoter and comprising an NHX-encoding gene. Also preferred is the use of a genetic construct comprising a weak constitutive promoter, such as the maize ubiquitin promoter with a deleted first intron and an NHX-encoding gene.

In the present invention, monocotyledonous plants are transformed with a genetic construct, such as an expression vector, comprising the sequence of interest operably linked to a promoter. Advantageously, any type of promoter may be used to drive expression of the nucleic acid sequence depending on the desired outcome. For example, a meristem-specific promoter, such as the RNR (ribonucleotide reductase), cdc2a promoter and the cyc07 promoter, could be used to effect expression in all growing parts of the plant, thereby increasing cell proliferation, which in turn increases yield, harvest index or biomass. If the desired outcome would be to (further) influence seed characteristics, such as the storage capacity, seed size, seed number, biomass etc., then a seed-specific promoter, such as p2S2, pPROLAMIN, pOLEOSIN could be selected. An aleurone-specific promoter may be selected in order to increase growth at the moment of germination, thereby increasing the transport of sugars to the embryo. An inflorescence-specific promoter, such as pLEAFY, may be utilised if the desired outcome would be to modify the number of flower organs. To produce male-sterile plants one would need an anther specific promoter. To impact on flower architecture for example petal size, one could choose a petal-specific promoter. If the desired outcome would be to modify growth and/or developmental characteristics in particular organs, then the choice of the promoter would depend on the organ to be modified. For example, use of a root-specific promoter would lead to increased growth and/or increased biomass or yield of the root and/or phenotypic alteration of the root. This would be particularly important where it is the root itself that is the desired end product, such crops including sugar beet, turnip, carrot, and potato. A fruit-specific promoter may be used to modify, for example, the strength of the outer skin of the fruit or to increase the size of the fruit. A green tissue-specific promoter may be used to increase leaf size. A cell wall-specific promoter may be used to increase the rigidity of the cell wall, thereby increasing resistance towards pathogens. An anther-specific promoter may be used to produce male-sterile plants. A vascular-specific promoter may be used to increase transport from laves to seeds. A nodule-specific promoter may be used to increase the nitrogen fixing capabilities of a plant, thereby increasing the nutrient levels in a plant. Also constitutive promoters, such as the GOS2 promoter, or promoters with a similar strong and ubiquitous expression pattern, may be useful in the methods of the present invention.

Excluded from the invention are transgenic plants described in Ohta et al. (FEBS Letters 532 (2002) 279-282). These plants contain the Na⁺/H⁺ antiporter gene from A gmelini (AgNHX1), which was amplified by PCR and digested with Sall and BamHI restriction enzymes. The resulting fragment was inserted between the promoter region and the terminator region of the expression vector pIG221 to produce p35S/I/AgNhx1, replacing the cDNA sequence of the GUS gene. The promoter region contains the cauliflower mosaic virus (CaMV) 35S promoter and the first intron of catalase from Ricinus communis L, while the terminator region contains the polyadenylation signal of the nopaline synthetase gene (Nos). Subsequently, the expression cassette from p35S/I/AgNhx1 was inserted into the plasmid pHSG398 (Takara Biomedicals, Japan) to produce pHSG/AgNhx1.

The terms “regulatory element”, “control sequence”, “promoter” are all used herein interchangeably and taken in a broad context refer to regulatory nucleic acid sequences capable of effecting expression of the sequences to which they are ligated. Encompassed by the aforementioned terms are transcriptional regulatory sequences derived from a classical eukaryotic genomic gene (including the TATA box which is required for accurate transcription initiation, with or without a CCAAT box sequence) and additional regulatory elements i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative, which confers, activates or enhances expression of a nucleic acid molecule in a cell, tissue or organ. The terms “control sequence”, “regulatory sequence”, “regulatory element” and “promoter” are used interchangeably herein. The term “operably linked” as used herein refers to a functional linkage between the promoter sequence and the gene of interest such that the promoter sequence is able to initiate transcription of the gene of interest.

Optionally, one or more terminator sequences may also be used in the construct introduced into a plant. The term “terminator” encompasses a control sequence which is a DNA sequence at the end of a transcriptional unit which signals 3′ processing and polyadenylation of a primary transcript and termination of transcription. Additional regulatory elements may include transcriptional as well as translational enhancers. Those skilled in the art will be aware of terminator and enhancer sequences, which may be suitable for use in performing the invention. Such sequences would be known or may readily be obtained by a person skilled in the art.

The genetic constructs of the invention may further include an origin of replication sequence which is required for maintenance and/or replication in a specific cell type, for example a bacterial cell, when said genetic construct is required to be maintained as an episomal genetic element (e.g. plasmid or cosmid molecule) in a cell. Preferred origins of replication include, but are not limited to, the f1-ori and colE1 origins of replication.

The genetic construct may optionally comprise a selectable marker gene. As used herein, the term “selectable marker gene” includes any gene which confers a phenotype on a cell in which it is expressed to facilitate the identification and/or selection of cells which are transfected or transformed with a genetic construct of the invention or a derivative thereof. Suitable markers may be selected from markers that confer antibiotic or herbicide resistance. Cells containing the recombinant DNA will thus be able to survive in the presence of antibiotic or herbicide concentrations that kill untransformed cells. Examples of selectable marker genes include the bar gene which provides resistance to the herbicide Basta; the neomycin phosphotransferase (nptII) gene which confers resistance to the antibiotic kanamycin; the hygromycin phosphotransferase (hptII) gene which confers hygromycin resistance. Visual markers, such as the Green Fluorescent Protein (GFP) (Haseloff et al., Proc. Natg. Acad. Sci. U.S.A, 94 (6), 2122-2127, 1997) and luciferase may also be used as selectable markers. An entire plant may be generated from a single transformed plant cell through cell culturing techniques known to those skilled in the art. Other suitable selectable marker genes include the ampicillin resistance (Amp′), tetracycline resistance gene (Tc′), bacterial kanamycin resistance gene (Kan′), phosphinothricin resistance gene, β-glucuronidase (GUS) gene, and chloramphenicol acetyltransferase (CAT) gene amongst others.

Recombinant DNA constructs for use in the methods according to the present invention may be constructed using recombinant DNA technology well known to persons skilled in the art. The gene constructs may be inserted into vectors, which may be commercially available, suitable for transforming into plants and suitable for expression of the gene of interest in the transformed cells.

The present invention also relates to a method for the production of transgenic monocotyledonous plants, plant cells or plant tissues, comprising introduction of a nucleic acid molecule of the invention in an expressible format or a vector as defined above into a plant, plant cell or plant tissue. The nucleic acid may be operably linked to one or more control sequences or may be integrated in a vector according to the invention and/or may be stably integrated into the genome of a plant cell.

The nucleic acid molecule or a genetic construct according to the invention comprising the NHX-encoding gene, may be introduced into a monocotyledonous plant cell using any known method for the transfection or transformation of a cell. A whole organism may be regenerated from a single transformed or transfected cell, using methods known in the art. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular spades being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem).

The gene of interest is preferably introduced into a plant by transformation. The term “transformation” as referred to herein encompasses the transfer of an exogenous polynucleotide into a host cell, irrespective of the method used for transfer. The polynucleotide may be transiently or stably introduced into a host cell and may be maintained non-integrated, for example, as a plasmid, or alternatively, may be integrated into the host genome. The resulting transformed plant cell can then be used to regenerate a transformed plant in a manner known to persons skilled in the art. Transformation of a plant species is now a fairly routine technique. Advantageously, any of several transformation methods may be used to introduce the gene of interest into a suitable ancestor cell. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., Nature 296, 72-74, 1982; Negrutiu I. et al., Plant Mol. Biol. 8, 363-373, 1987); electroporation of protoplasts (Shillito R. D. et al. Bio/Technol 3, 1099-1102, 1985); microinjection into plant material (Crossway A et al., Mol. Gen. Genet 202, 179-185, 1986); DNA or RNA-coated particle bombardment (Klein T. M. et al., Nature 327, 70, 1987) infection with (non-integrative) viruses and the like. A preferred method according to the present invention comprises the protocol according to Hiei et al., Plant J., 6 (2), 271-282, 1994 in the case of rice transformation. For corn transformation, a preferred method according to the present invention, comprises the Agrobacterium-based transformation of an immature embryo as described in EP0604662, EP0672752, EP0971578, EP0955371 or EP0558676 or following the protocol of Frame et al. (Agrobacterium tumefaciens-mediated transformation of mare embryos using a standard binary vector system. Plant Physiol. 2002 May; 129(1):13-22).

Generally after transformation, plant cells or cell groupings are selected for the presence of one or more markers which are encoded by plant-expressible genes co-transferred with the gene of interest, following which the transformed material is regenerated into a whole plant. Following DNA transfer and regeneration, putatively transformed plants may be evaluated, for instance using Southern analysis, for the presence of the gene of interest, copy number and/or genomic organisation. Alternatively or additionally, expression levels of the newly introduced DNA may be undertaken using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed to give homozygous second generation (or T2) transformants, and the T2 plants further propagated through classical breeding techniques.

The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention also provides monocotyledonous plants obtainable by the methods according to the invention, which plants have improved growth characteristics when compared to corresponding wild-type plants.

The present invention also provides a method for the production of a transgenic monocotyledonous plant having improved growth characteristics, which method comprises:
(i) introducing into a plant cell a nucleic acid encoding an NHX protein;
(ii) cultivating said plant cell under conditions promoting regeneration and mature plant growth.

Preferably, the NHX gene is under the control of a tissue-specific promoter, preferably an endosperm-specific promoter or a weak constitutive promoter, such as the maize ubiquitin promoter with deleted first intron.

The invention also provided host cells containing an isolated nucleic acid molecule encoding a NHX protein, preferably wherein the protein is a plant NHX protein, such as one of the aforementioned plant-derived proteins. Preferably, in these host cells, the NHX encoding gene is in the sense orientation and under the control of a tissue specific promoter, preferably an endosperm-specific promoter or a weak constitutive promoter, with as the maize ubiquitin promoter with deleted first intron.

The present invention extends to any transgenic monocotyledonous plant with improved growth characteristics containing an isolated nucleic acid molecule encoding an NHX protein. Preferred host cells according to the invention are monocotyledonous plant cells. The present invention extends to any plant or plant cell produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced in the parent by the methods according to the invention. The invention also extends to harvestable parts of a monocotyledonous plant, such as but not limited to seeds, leaves, flowers, fruits, stems, rhizomes, roots, and bulbs.

The term “plant” as used herein encompasses whole plants, ancestors and progeny of the plants and plant parts, including seeds, shoots, stems, roots and plant cells, tissues, organs, suspension cultures, embryos, meristematic regions, callus tissue, leaves, gametophytes, sporophytes, pollen, and microspores.

Plants that are particularly useful in the methods of the invention include sugarcane or cereals such as rice, maze, wheat barley, millet, rye, oats or sorghum.

Advantageously, performance of the methods according to the present invention leads to plants having improved plant growth characteristics and/or having various improved growth characteristics, such as improved (modified) growth, improved yield/biomass and improved architecture, each relative to the corresponding wild-type plants and when grown under non-salt stress conditions.

The term “improved plant growth” as used herein encompasses, but is not limited to, a faster rate of growth in one or more parts of a plant (including green biomass and including seeds), at one or more stages in the life cycle of a plant, and/or enhanced vigour, each relative to corresponding wild-type plants and grown under non-salt stress growing conditions.

The term “modified yield”, preferably “increased yield”, encompasses an increase in biomass in one or more parts of a plant relative to the biomass of corresponding wild-type plants and when grown under non-salt stress growing conditions. The term also encompasses an increase in seed yield, which includes an increase in the biomass of the seed and/or an increase in the number of (filled) seeds and/or in the size of the seeds and/or an increase in seed volume, each relative to corresponding wild-type plants and when grown under non-salt stress growing conditions. An increase in seed size and/or volume may also influence the composition of the seed, for example, they may contain more starch or more oil and grown and produced under non-salt stress going conditions. An increase in seed yield could be due to an increase in the number and/or size of flowers. An increase in seed yield might also increase the harvest index, which is expressed as the ratio of the total biomass over the yield of harvestable parts, such as the seeds of a cereal. Increased yield/biomass refers to a better performance of a plant under non-salt stress conditions compared to the performance of a wild-type plant.

According to a preferred feature of the present invention, performance of the methods according to the present invention result in plants having modified yield. Preferably the modified yield includes, an increase in number of filled seeds, an increase in total number of seeds, an increase in the total seed weight, an increase in thousand kernel weight, and increase in harvest index and an increase in the number of panicles each relative to the corresponding wild-type plants.

Therefore, according to the invention, there is provided a method for increasing yield of monocotyledonous plants, which method comprises increasing expression of a nucleic acid encoding an NHX protein and/or modulating the activity of an NHX protein in a plant.

The methods of the present invention are particularly favourable to be applied to crop plants, preferably cereals, because the methods of the present invention are used to increase the seed yield, thousand kernel weight and harvest index of the plant. Accordingly, a particular embodiment of the present invention relates to a method to increase seed yield and/or to increase harvest index of a cereal, comprising increasing expression of a nucleic acid encoding an NHX protein and/or modulating the activity of an NHX protein in a plant.

The term “architecture” as used herein encompasses the appearance or morphology of a plant including any one or more structural features or combination of structural features thereof. Such structural features include the shape, size, number, position, texture, arrangement, and pattern of any cell, issue or organ or groups of cells, tissues or organs of a plant including the root, leaf, shoot stem, petiole, trichome, flower, petal, stigma, style, stamen, pollen, ovule, seed, embryo, endosperm, seed coat, aleurone, fibre, cambium, wood, heartwood, parenchyma, aerenchyma, sieve element phloem or vascular tissue, amongst others. Modified architecture therefore includes all aspects of modified growth of the plant.

The present invention relates to methods to improving growth characteristics of a plant or methods to produce plants with improving growth characteristics, wherein the growth characteristics comprise any one or more selected from: increased yield, Increased biomass, Increased total above ground area, increased plant height increased number of tillers, increased number of first panicles, increased number of second panicles, increased number of filled seeds, increased total seed yield per plant, increased harvest index, increased thousand kernel weight, increased Tmid, increased T45 or t90, increased A42 or an altered growth curve.

Also the present invention provides methods to alter one of the above mentioned growth characteristics, without causing a penalty on one of the other growth characteristics, for example increased the above ground green issue area while retaining the same number of filled seeds and the same seed yield.

The invention also relates to use of a nucleic acid encoding an NHX protein and/or use of the NHX protein itself in improving the growth characteristics of monocotyledonous plants grown under non-salt stressed conditions.

The growth characteristics preferably include increased yield/biomass, modified architecture, increased seed yield and increased harvest index.

Those skilled in the art will be aware that the invention described herein is subject to variations and modifications other than those specifically described. It is to be understood that the invention described herein includes all such variations and modifications. The invention also includes all such steps, features, compositions and compounds referred to or indicated in this specification, individually or collectively, and any and all combinations of any or more of said steps or features.

The present invention will now be described with reference to the following figures in which:

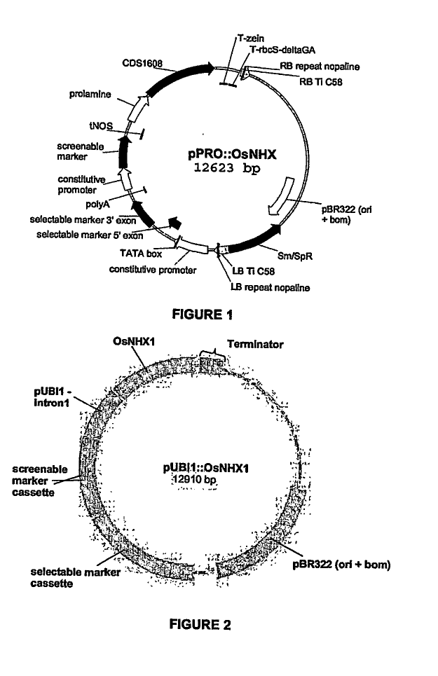

FIG. 1 is a map of a plasmid containing the Oryza saliva sequence CDS1608 which is the internal code for the cDNA encoding an NHX protein under control of the rice prolamin promoter (internal code PRO0090), the double terminator sequence T-zein and T-rbcS-deltaGA, located within the borders (the left border (LB repeat, LB Ti C58) and the right border (RB repeat, RB Ti C58)) of the nopaline Ti plasmid; a screenable marker and a selectable marker both cloned within the T-borders and each under a constitutive promoter and each followed by a terminator sequence tNOS and/or a poly A tail; origin of replication (pBR322 (ori+bom)) and a bacterial selectable marker (Sm/SpR).

FIG. 2 is a map of a plasmid containing the Oryza sativa sequence CDS1608 which is the internal code for the cDNA encoding an NIX protein, under the control of the maize ubiquitin promoter with deleted first intron (internal code PRO0221), the double terminator sequence T-zein and T-rbc-S-deltaGA, located within the borders (the left border (LB repeat, LB Ti C58) and the right border (RB repeat, RB Ti C58)) of the nopaline Ti plasmid; a screenable marker and a selectable marker both cloned within the T-borders and each under a constitutive promoter and each followed by a terminator sequence tNOS and/or a poly A tail; origin of replication (pBR322 (ori+bom)) and a bacterial selectable marker (Sm/SpR).

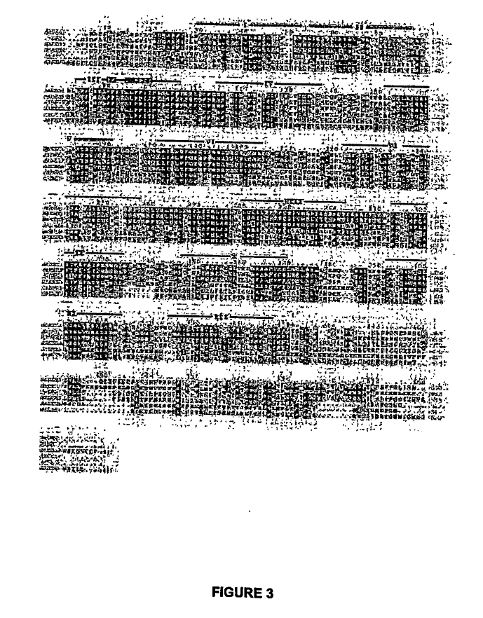

FIG. 3 is an alignment from Yokoi et al., 2002 showing six Arabidopsis thaliana isogenes (AtNHX) encoding proteins that have domains with sequence similarity to metazoan NHE Na+/H+ exchangers. The predicted peptides from AtNHX1 (AF106324), AtNHX2 (AC009465), AtNHX3 (AC0011623), AtNHX4 (AB015479). AtNHX5 (AC005287) and AtNHX6 (AC010793) are aligned based on analysis using the CLUSTAL V method (Higgins and Sharp, 1989). Residues that are identical in all AtNHX family members are highlighted in black, those identical in subgroup 1 family members (AtNHX1±4) are shaded in dark grey, and those residues speci [symbol] /Ec to the subgroup 2 family members (AtNHX5 and 6) are highlighted in light grey. Putative transmembrane domains are indicated by Roman numerals. The consensus amiloride binding motif is indicated in transmembrane domain III.

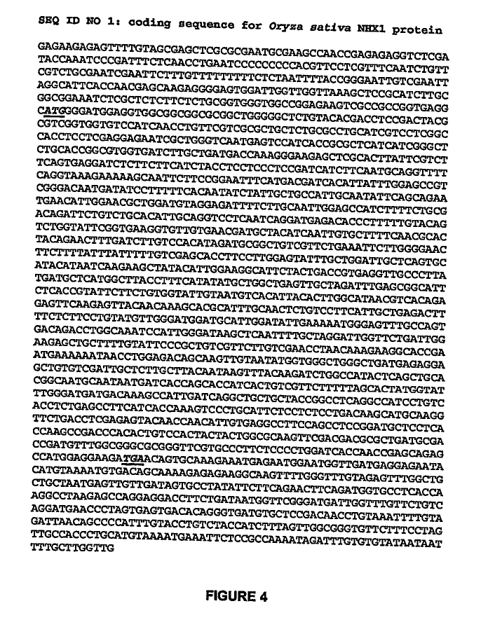

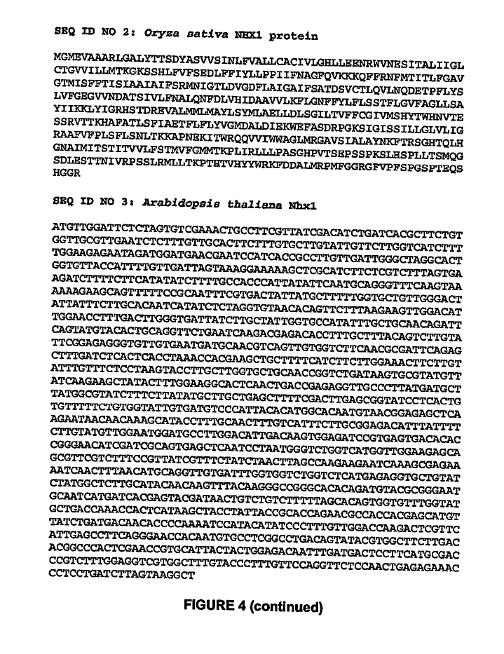

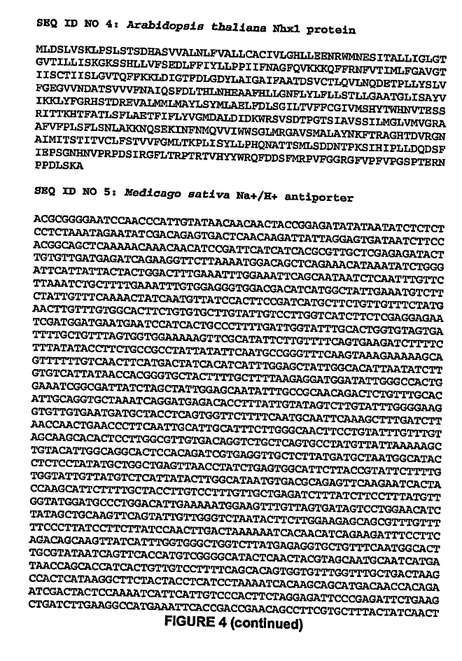

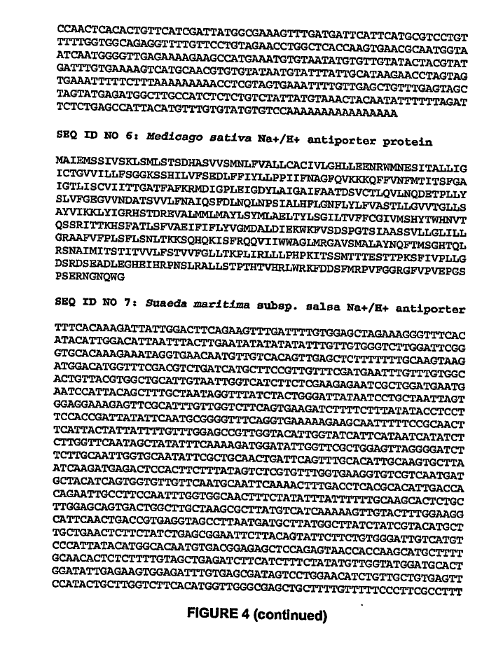

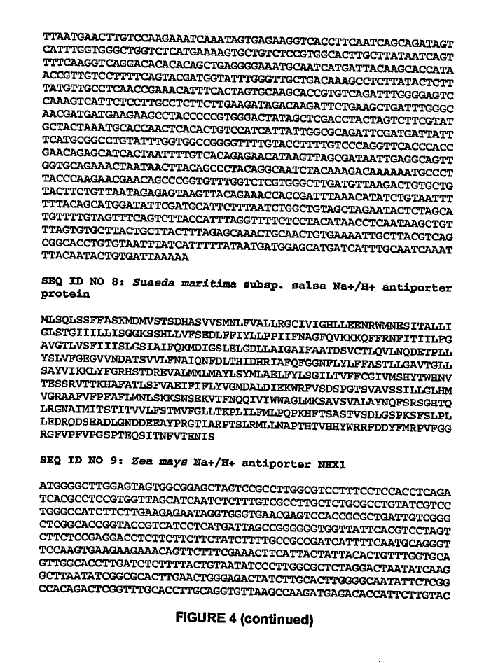

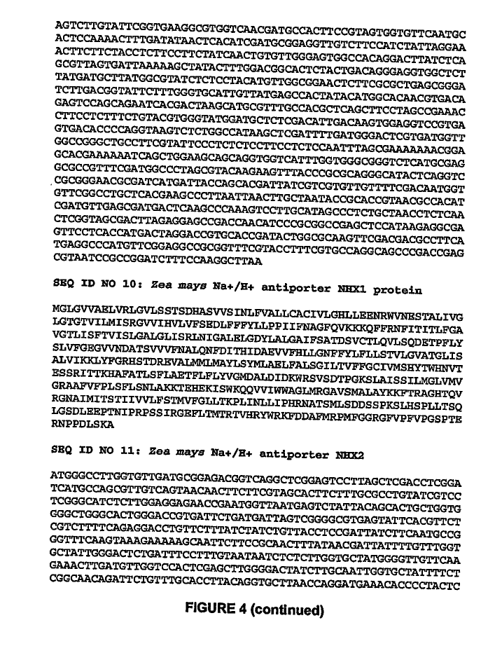

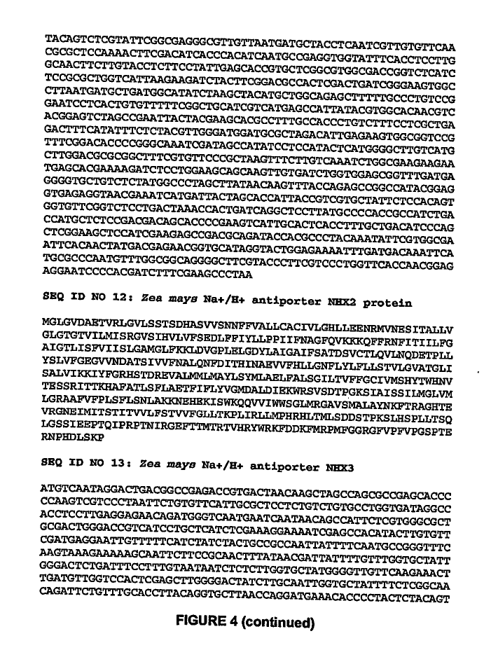

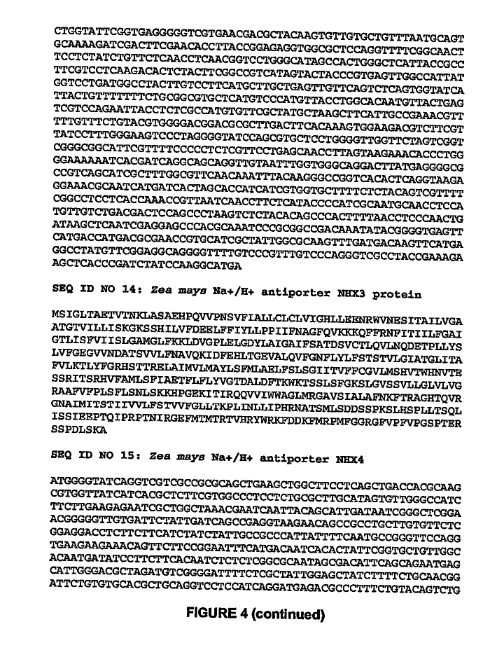

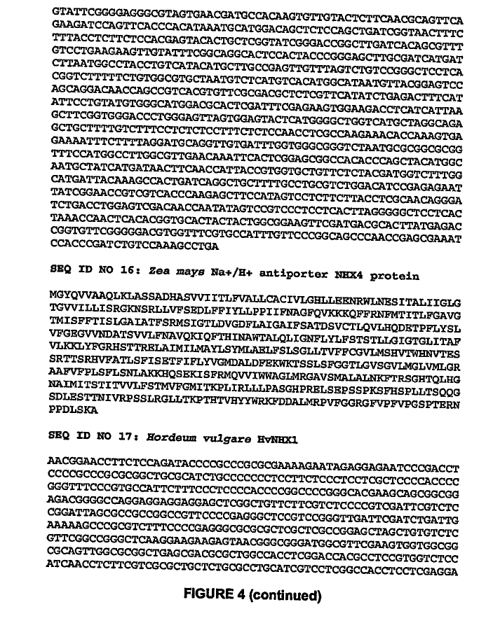

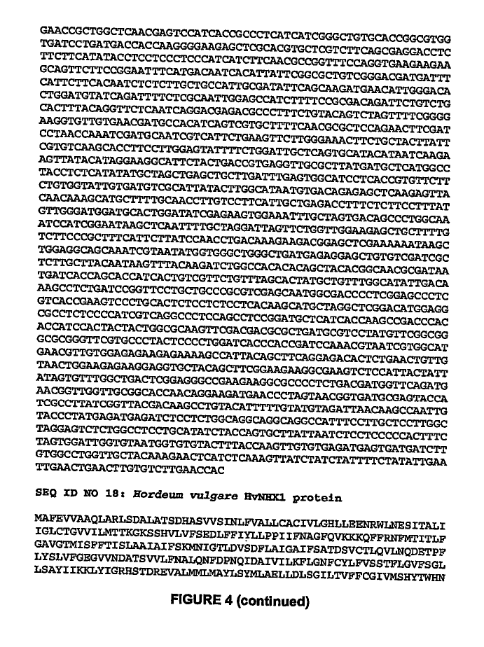

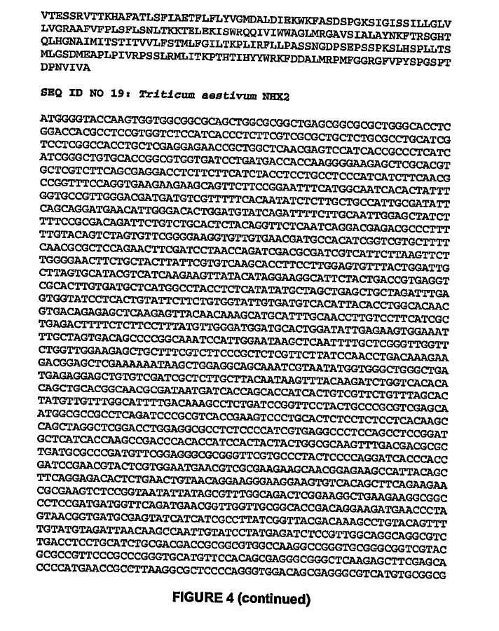

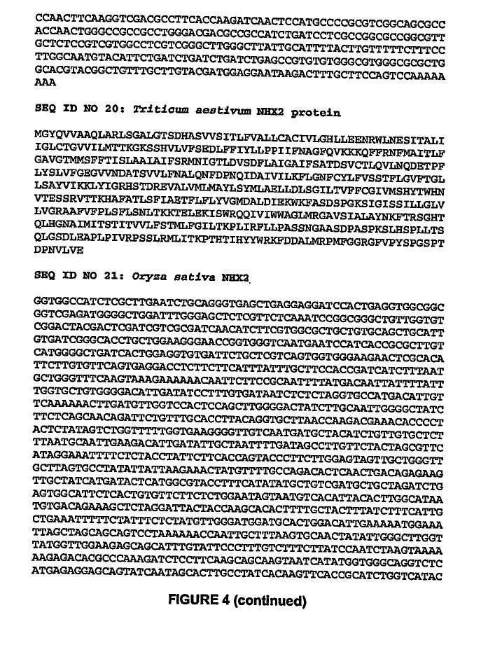

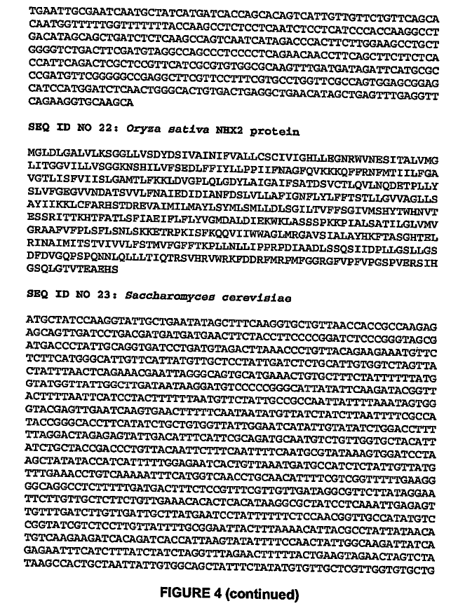

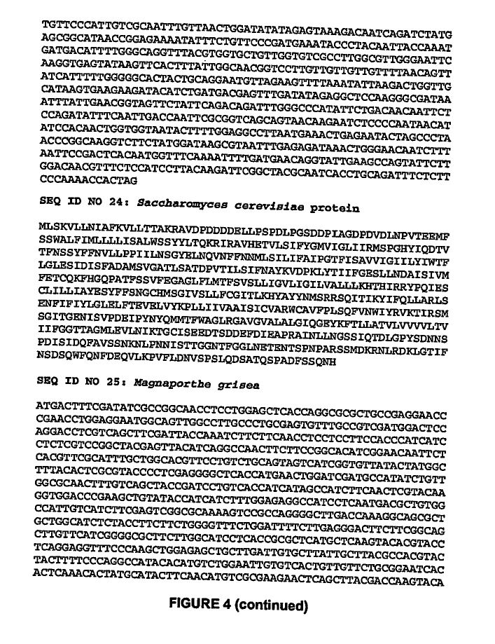

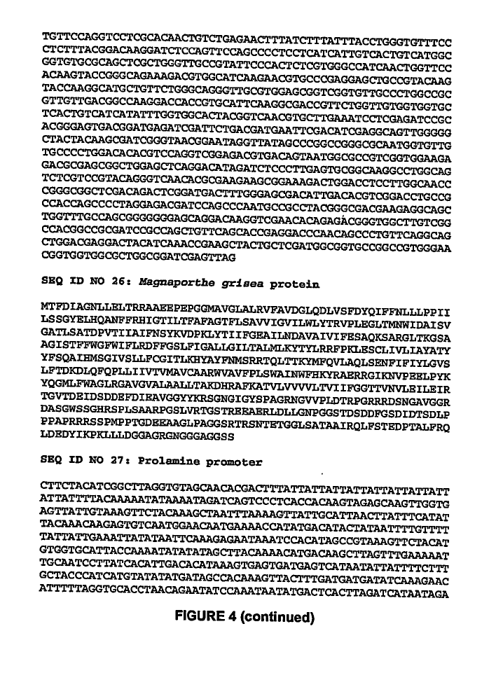

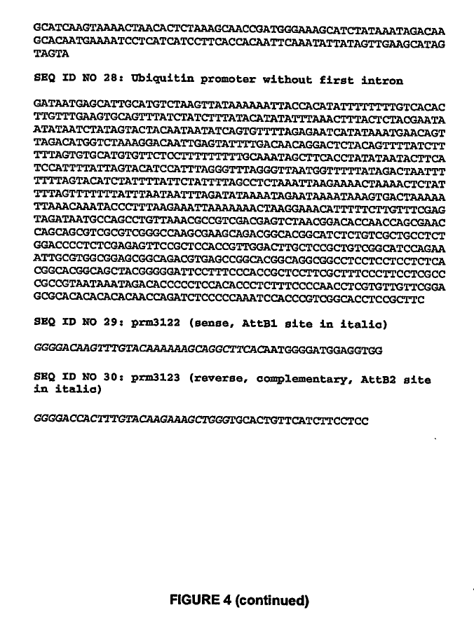

FIG. 4 lists examples of sequences useful in the methods of the invention.

### EXAMPLES

The present invention will now be described with reference to the following examples, which are by way of illustration alone.

DNA Manipulation

Unless otherwise stated, recombinant DNA techniques are performed according to standard protocols described in (Sambrook (2001) Molecular Cloning: a laboratory manual, 3rd Edition Cold Spring Harbor Laboratory Press, CSH, New York) or in Volumes 1 and 2 of Ausubel et al., (Current Protocols in Molecular Biology. N.Y. John Wiley and Sons, 1998). Standard materials and methods for plant molecular work are described in Plant Molecular Biology Labfax (1993) by R. D. D. Cray, published by BIOS Scientific Publications Ltd (UK) and Blackwell Scientific Publications (UK).

### Example 1

### Cloning the Rice NHX Gene (CDS1608, SEQ ID NO 1)

The rice gene encoding NHX1 (CDS1608) was amplified by PCR using as a template an Oryza saliva japonica cv Nipponbare seedling cDNA library (Invitrogen, Paisley, UK). After reverse transcription of RNA extracted from seedlings, the cDNAs were cloned into pCMV Sport 6.0. Average insert size of the bank was 1.5 kb, and the original number of clones was of 1.59×10⁷ cfu. Original titer was determined to be 9.6×10⁵ cfu/ml, after a first amplification of 6×10¹¹ cfu/ml. After plasmid extraction, 200 ng of template was used in a 50 μl PCR mix. Primers prm3122 (sense, AttB1 site in italic: 5′-GGGGACAAGTTTGTACAAAAAAGCAGGCTTCACAATGGGGATGGAGGTGG-3′) (SEQ ID NO 30) and prm3123 (reverse, complementary, AttB2 site in italic: 5′-GGGGACCACTTTGTACAAGAAAGCTGGGTGCACTGTTCATCTTCCTCC-3′) (SEQ ID NO 31), which include the AttB sites for Gateway recombination, were used for PCR amplification. PCR was performed using Hifi Taq DNA polymerase under standard conditions. A PCR fragment of 1608 bp was amplified and purified also using standard methods. The first step of the Gateway procedure, the BP reaction, was then performed, during which the PCR fragment was recombined in vivo with the pDONR201 plasmid to produce, according to the Gateway terminology, an “entry clone”. Plasmid pDONR201 was purchased from Invitrogen, as part of the Gateway® technology.

The entry done was subsequently used in an LR reaction with p0830, a destination vector used for Oryza sativa transformation. This vector contains as functional elements within the T-DNA borders: a plant selectable marker expression cassette; a screenable marker expression cassette; a Gateway cassette intended for LR in vivo recombination with the sequence of interest already cloned in the entry done. A rice prolamin promoter (PRO0090) for expression of the NHX gene was located upstream of this Gateway cassette.

After the LR recombination step, the resulting expression vector (FIG. 1) was transformed into Agrobacterium strain LBA4044 and subsequently into Oryza sativa plants.

In a second experiment, the rice NHX1 coding sequence was cloned in another destination vector, p02416, which comprised the same functional elements within its T-DNA borders, except that the promoter was a maize ubiquitin promoter without intron, instead of the rice GOS2 promoter. After the LR recombination step, the resulting expression vector p0494 (FIG. 2) was transformed into Agrobacterium strain LBA4044 and subsequently into Oryza saliva plants.

Transformed rice plants were allowed to grow and were then examined for the parameters described in Example 3.

### Example 2

### Evaluation of Transgenic Rice Transformed with Prolamin::OsNKX1 and Ubiquitin Minus Intron 1:: OsNHX1

Approximately 15 to 20 independent T0 rice transformants were generated. The primary transformants were transferred from tissue culture chambers to a greenhouse for growing and harvest of T1 seed. 5 to 6 events, of which the T1 progeny segregated 3:1 for presence/absence of the transgene, were retained. For each of these events, approximately 10 T1 seedlings containing the transgene (hetero- and homo-zygotes), and approximately 10 T1 seedlings lacking the transgene (nullizygotes), were selected by monitoring expression of the screenable marker.

The selected T1 plants (approximately 10 with the transgene and approximately 10 without the transgene) were transferred to a greenhouse. Each plant received a unique barcode label to link unambiguously the phenotyping data to the corresponding plant. The selected T1 plants were grown in sod in 10 cm diameter pots under the following environmental settings: photoperiod=11.5 h, daylight intensity=30,000 lux or more, daytime temperature=28° C. or higher, nighttime temperature=22° C., relative humidity=60-70%. Transgenic plants and the corresponding nullizygotes were grown side-by-side at random positions. From the stage of sowing until the stage of maturity each plant were passed several times through a digital imaging cabinet and imaged. At each time point digital images (2048×1536 pixels, 16 million colors) were taken of each plant from at least 6 different angles.

The mature primary panicles were harvested, bagged, barcode-labelled and then dried for three days in the oven at 37° C. The panicles were then threshed and all the seeds collected. The filed husks were separated from the empty ones using an air-blowing device. After separation, both seed lots were then counted using a commercially available counting machine. The empty husks were discarded. The filled husks were weighed on an analytical balance and the cross sectional area of the seeds was measured using digital imaging. This procedure resulted in the set of seed-related parameters described below.

These parameters were derived in an automated way from the digital images using image analysis software and were analysed statistically. A two factor ANOVA (analyses of variance) corrected for the unbalanced design was used as statistical model for the overall evaluation of plant phenotypic characteristics. An F-test was carried out on all the parameters measured of all the plants of all the events transformed with that gene. The F-test was carried out to check for an effect of the gene over all the transformation events and to verify for an overall effect of the gene, also named herein “global gene effect”. If the value of the F test showed the data to be significant it was concluded that there was a “gene” effect, meaning that not only presence or the position of the gene is causing the effect. The threshold for significance for a true global gene effect is set at 5% probability level for the F test.

Vegetative growth and seed yield were measured according to the methods as described above. The inventors surprisingly found that the Thousand Kernel Weight, the number of filled seeds, the total weight of seeds harvested, the harvest index, biomass and plant height were all increased in rice plants transformed with an OsNHX1 gene compared to control plants without the OsNHX1 gene.

The data obtained in a first experiment for the PRO0221::CDS1608 construct were confirmed in a second experiment with T2 plants. Three lines were selected for further analysis. Seed batches from the positive plants (both hetero- and homozygotes) in T1, were screened by monitoring marker expression. For each chosen event, the heterozygote seed batches were then retained for T2 evaluation. Within each seed batch an equal number of positive and negative plants were grown in the greenhouse for evaluation. A total number of 120 OsNHX1 transformed plants were evaluated in the T2 generation, that is, 40 plants per event of which 20 were positives for the transgene and 20 negative.

### Example 3

### Results of the Evaluation of Transgenic Plants Transformed with PRO0090::CDS1608 or PRO0221:: CDS1608

Vegetative growth and seed yield were measured according to the methods as described above.

Evaluation of the PRO0090::CDS1608 Construct

In this first experiment, the inventors demonstrated that the harvest index and the Thousand Kernel Weight (TKW) were increased in the transgenic plants with the PRO0090::CDS1608 construct (5 events tested) compared to corresponding nullizygous plants. The harvest index is herein defined as the ratio between the total seed yield and the above ground area (mm²), multiplied by a factor 106. The TKW of plants is derived from the number of filed seeds counted and their total weight. Statistical analysis showed that the measured increases for both parameters were significant. For the harvest index and taking all the tested lines into account, an overall increase of 19% was found with a p-value for the F-test of 0.0428. Similarly, an overall increase of the TKW of 4% was measured (p-value of the F-test 0.0337). These data show that there was a global gene effect causing an increase in harvest index and TKW.

Evaluation of the PRO0221::CDS1608 Construct

In a second experiment, transgenic plants carrying the PRO0221::CDS1608 construct were analysed. In an initial experiment, plants from 6 independent events of transformation were analysed at the T1 generation as previously described. An increase in plant height, harvest index, number of filled seeds, seed fill rate (number of seeds filled over the total harvested) were observed for the transgenic plants when compared to their corresponding nullizygous. The positive effect being present in individual events and in some cases with a global gene effect. Three events were selected for further analysis in the following generation, the T2 generation. The analysis involved a greater number of plants per event and was performed as described above. Data indicated that the transgenic plants had an overall increase in harvest index and seed fill rate, strongly suggesting a global gene effect on the set of events analysed. Additionally, transgenic plants (when compared to their corresponding nullizygotes) of individual events showed a significant increase in total area (biomass) in number of filled seeds and total seed weight.

Table 3 below shows data for two parameters showing global gene effects over all the events tested: harvest index and seed filled rate.

TABLE 3

| | T1 plants | | T2 plants | |
|---|---|---|---|---|
| parameter | % increase | p-value | % increase | p-value |
| Harvest Index | 20 | 0.0112 | 10 | 0.0817 |
| Seed fill rate (*) | 21 | 0.0114 | 10 | 0.0717 |

(*) Seed fill rate calculated as the ratio between the number of silled seeds over the total number of harvested seeds.

Number of Filled Seeds

The number of filled seeds was determined by counting the number of filled husks that remained after the separation step. All 6 tested lines showed an increase in filled seed number, amounting to over 33%. There was an overall increase of 14% in the number of filled seeds produced by transgenic plants relative to corresponding null segregants. In the T2 generation, there was a mean increase 15%, this mean increase was also statistically significant (p-value of 0.0433). The combined analysis of T1 and T2 data also confirmed that the global gene effect was highly significant (p-value of 0.0086).

Harvest Index

The harvest index in the present invention is defined as the ratio between the total seed yield and the above ground area (mm²), multiplied by a factor 10⁶. All six tested lines showed an increased harvest index, ranging between 1.6 and 18.26%. There was a significant overall gene effect (an effect associated with of the presence of the transgene) on harvest index (an overall increase of 20%), with a statistically significant p-value for the F test of 0.0112. Similar results were obtained for T2 plants. On average, the harvest index was increased with 10% (p-value of 0.0817). Here too, the combined analysis of the T1 and T2 data showed a global gene effect (p-value 0.0064).

1-21. (canceled)

22. A method for improving plant growth characteristics, comprising increasing, in a monocotyledonous plant, expression of a nucleic acid encoding an NHX protein and/or increasing activity of an NHX protein, wherein said plant is grown under non-salt stress conditions.

23. The method according to claim 22, wherein said increasing expression and/or increasing activity is affected by introducing and expressing in a plant a nucleic acid encoding an NHX protein or a homologue, derivative or active fragment thereof.

24. The method according to claim 22, wherein said growth characteristic is increased yield/biomass and/or modified plant architecture.

25. The method according to claim 24, wherein said increased yield/biomass and/or modified plant architecture is selected from the group consisting of: increased aboveground area, increased number of first panicles, increased plant height, increased total number of seeds, increased number of filled seeds, increased total seed weight, increased harvest index and increased thousand kernel weight, each relative to corresponding wild type plants grown under non-salt stress conditions.

26. The method according to claim 22, wherein said nucleic acid is in the sense orientation and is under the control of a tissue-specific promoter.

27. The method according to claim 22, wherein said nucleic acid is in the sense orientation and is under the control of a seed-specific promoter.

28. The method according to claim 22, wherein said nucleic acid is in the sense orientation and is under the control of an endosperm-specific promoter such as a prolamin promoter.

29. The method according to claim 22, wherein said nucleic acid is in the sense orientation and is under the control of a weak constitutive promoter such as a maize ubiquitin promoter minus first intron.

30. The method according to claim 22, wherein said nucleic acid is from a monocotyledonous plant from the family Poaceae.

31. The method according to claim 22, wherein said nucleic acid is from a monocotyledonous plant from the family Poaceae and from the genus Oryza.

32. The method according to claim 22, wherein said nucleic acid is represented by SEQ ID NO: 1 or a portion thereof or a sequence capable of hybridizing therewith.

33. Plants obtainable by the method of claim 22, which plants have improved growth characteristics relative to corresponding wild type plants.

34. A method for the production of a monocotyledonous plant having improved growth characteristics, which method comprises the steps of:
(i) introducing and expressing, in a plant, a nucleic acid encoding an NHX protein or a homologue, derivative or active fragment thereof; and
(ii) cultivating said plant under non-salt stress conditions.

35. Plants obtainable by the method according to claim 34, which plants have improved growth characteristics relative to corresponding wild type plants, wherein said plants are selected from the group consisting of rice, maize, wheat, barley, millet, rye, oats and sorghum.

36. Transgenic monocotyledonous plants grown under non-salt stress conditions, said plants having improved growth characteristics, and which plants contain an isolated nucleic acid molecule encoding an NHX protein, wherein said transgenic monocotyledonous plants are selected from the group consisting of rice, maize, wheat, barley, millet, rye, oats and sorghum.

37. A plant part, such as a harvestable plant part, a propagule or progeny from a plant, according to claim 35, wherein said plant part is selected from the group consisting of rice, maize, wheat, barley, millet, rye, oats and sorghum.

38. A plant part, such as a harvestable plant part, a propagule or progeny from a plant, according to claim 36, wherein said plant part is selected from the group consisting of rice, maize, wheat, barley, millet, rye, oats and sorghum.

39. Harvestable parts according to claim 32, wherein said harvestable parts are seeds.

40. The plant according to claim 35 selected from sugarcane or a cereal such as rice, maize, wheat, barley, millet, rye, oats and sorghum.

41. The method of claim 22, further comprising administering a genetic construct to the monocotyledonous plant, wherein the genetic construct comprises:
a) a nucleic acid encoding an NHX protein;
b) a control sequence capable of regulating expression of the nucleic acid of (a); and optionally,
c) a transcription termination sequence.

42. The method according to claim 41, wherein said control sequence of (b) comprises a seed-specific promoter.

43. The method according to claim 41, wherein said control sequence of (b) comprises an endosperm-specific promoter.

44. The method according to claim 41, wherein said control sequence of (b), comprises a weak constitutive promoter such as a maize ubiquitin promoter with deleted first intron.

45. The method according to claim 41, wherein said nucleic acid is from a monocotyledonous plant from the family Poaceae.

46. The method according to claim 41, wherein said nucleic acid is from a monocotyledonous plant from the family Poaceae and from the genus Oryza.

47. The method according to claim 41, wherein said nucleic acid is represented by SEQ ID NO: 1 or a portion thereof or a sequence capable of hybridizing therewith.

48. A method for improving the growth characteristics of a monocotyledonous plant grown under non-salt stress conditions, comprising administering a nucleic acid encoding an NHX protein and/or an NHX protein to the monocotyledonous plant.

49. The method according to claim 48, wherein said growth characteristics comprise increased yield/biomass and/or altered architecture.

50. The method according to claim 48, wherein said nucleic acid encodes an NHX1 protein.

51. A method for breeding monocotyledonous plants in breeding programs grown under non-salt stress conditions, comprising administering a nucleic acid encoding an NHX protein and/or an NHX protein to the monocotyledonous plants.


SEQUENCE LISTING (30 sequences)

SEQ ID NO: 1 - DNA, 2313 bp, Oryza sativa
gagaagagagttttgtagcgagctcgcgcgaatgcgaagccaaccgagagaggtctcgataccaaatcccgatttctcaacctgaatcccccccccacgttcctcgtttcaatctgttcgtctgcgaatcgaattctttgtttttttttctctaattttaccgggaattgtcgaattaggcattcaccaacgagcaagaggggagtggattggttggttaaagctccgcatcttgcggcggaaatctcgctctcttctctgcggtgggtggccggagaagtcgccgccggtgaggcatggggatggaggtggcggcggcgcggctgggggctctgtacacgacctccgactacgcgtcggtggtgtccatcaacctgttcgtcgcgctgctctgcgcctgcatcgtcctcggccacctcctcgaggagaatcgctgggtcaatgagtccatcaccgcgctcatcatcgggctctgcaccggcgtggtgatcttgctgatgaccaaagggaagagctcgcacttattcgtcttcagtgaggatctcttcttcatctacctcctccctccgatcatcttcaatgcaggttttcaggtaaagaaaaagcaattcttccggaatttcatgacgatcacattatttggagccgtcgggacaatgatatcctttttcacaatatctattgctgccattgcaatattcagcagaatgaacattggaacgctggatgtaggagattttcttgcaattggagccatcttttctgcgacagattctgtctgcacattgcaggtcctcaatcaggatgagacaccctttttgtacagtctggtattcggtgaaggtgttgtgaacgatgctacatcaattgtgcttttcaacgcactacagaactttgatcttgtccacatagatgcggctgtcgttctgaaattcttggggaacttcttttatttatttttgtcgagcaccttccttggagtatttgctggattgctcagtgcatacataatcaagaagctatacattggaaggcattctactgaccgtgaggttgcccttatgatgctcatggcttacctttcatatatgctggctgagttgctagatttgagcggcattctcaccgtattcttctgtggtattgtaatgtcacattacacttggcataacgtcacagagagttcaagagttacaacaaagcacgcatttgcaactctgtccttcattgctgagacttttctcttcctgtatgttgggatggatgcattggatattgaaaaatgggagtttgccagtgacagacctggcaaatccattgggataagctcaattttgctaggattggttctgattggaagagctgcttttgtattcccgctgtcgttcttgtcgaacctaacaaagaaggcaccgaatgaaaaaataacctggagacagcaagttgtaatatggtgggctgggctgatgagaggagctgtgtcgattgctcttgcttacaataagtttacaagatctggccatactcagctgcacggcaatgcaataatgatcaccagcaccatcactgtcgttctttttagcactatggtatttgggatgatgacaaagccattgatcaggctgctgctaccggcctcaggccatcctgtcacctctgagccttcatcaccaaagtccctgcattctcctctcctgacaagcatgcaaggttctgacctcgagagtacaaccaacattgtgaggccttccagcctccggatgctcctcaccaagccgacccacactgtccactactactggcgcaagttcgacgacgcgctgatgcgaccgatgtttggcgggcgcgggttcgtgcccttctcccctggatcaccaaccgagcagagccatggaggaagatgaacagtgcaaagaaatgagaatggaatggttgatgaggagaatacatgtaaaatgtgacagcaaaagagagaaggcaagttttgggtttgtagagtttggctgctgctaatgagttgttgatagtgcctatattcttcagaacttcagatggtgcctcaccaaggcctaagagccaggaggaccttctgataatggttcgggatgattggtttgttctgtcaggatgaaccctagtgagtgacacagggtgatgtgctccgacaacctgtaaattttgtagattaacagccccatttgtacctgtctaccatctttagttggcgggtgttctttcctagttgccaccctgcatgtaaaatgaaattctccgccaaaatagatttgtgtgtataataattttgcttggttg

SEQ ID NO: 2 - protein, 535 aa, Oryza sativa
MGMEVAAARLGALYTTSDYASVVSINLFVALLCACIVLGHLLEENRWVNESITALIIGLCTGVVILLMTKGKSSHLFVFSEDLFFIYLLPPIIFNAGFQVKKKQFFRNFMTITLFGAVGTMISFFTISIAAIAIFSRMNIGTLDVGDFLAIGAIFSATDSVCTLQVLNQDETPFLYSLVFGEGVVNDATSIVLFNALQNFDLVHIDAAVVLKFLGNFFYLFLSSTFLGVFAGLLSAYIIKKLYIGRHSTDREVALMMLMAYLSYMLAELLDLSGILTVFFCGIVMSHYTWHNVTESSRVTTKHAFATLSFIAETFLFLYVGMDALDIEKWEFASDRPGKSIGISSILLGLVLIGRAAFVFPLSFLSNLTKKAPNEKITWRQQVVIWWAGLMRGAVSIALAYNKFTRSGHTQLHGNAIMITSTITVVLFSTMVFGMMTKPLIRLLLPASGHPVTSEPSSPKSLHSPLLTSMQGSDLESTTNIVRPSSLRMLLTKPTHTVHYYWRKFDDALMRPMFGGRGFVPFSPGSPTEQSHGGR

SEQ ID NO: 3 - DNA, 1614 bp, Arabidopsis thaliana
atgttggattctctagtgtcgaaactgccttcgttatcgacatctgatcacgcttctgtggttgcgttgaatctctttgttgcacttctttgtgcttgtattgttcttggtcatcttttggaagagaatagatggatgaacgaatccatcaccgccttgttgattgggctaggcactggtgttaccattttgttgattagtaaaggaaaaagctcgcatcttctcgtctttagtgaagatcttttcttcatatatcttttgccacccattatattcaatgcagggtttcaagtaaaaaagaagcagtttttccgcaatttcgtgactattatgctttttggtgctgttgggactattatttcttgcacaatcatatctctaggtgtaacacagttctttaagaagttggacattggaacctttgacttgggtgattatcttgctattggtgccatatttgctgcaacagattcagtatgtacactgcaggttctgaatcaagacgagacacctttgctttacagtcttgtattcggagagggtgttgtgaatgatgcaacgtcagttgtggtcttcaacgcgattcagagctttgatctcactcacctaaaccacgaagctgcttttcatcttcttggaaacttcttgtatttgtttctcctaagtaccttgcttggtgctgcaaccggtctgataagtgcgtatgttatcaagaagctatactttggaaggcactcaactgaccgagaggttgcccttatgatgcttatggcgtatctttcttatatgcttgctgagcttttcgacttgagcggtatcctcactgtgtttttctgtggtattgtgatgtcccattacacatggcacaatgtaacggagagctcaagaataacaacaaagcatacctttgcaactttgtcatttcttgcggagacatttattttcttgtatgttggaatggatgccttggacattgacaagtggagatccgtgagtgacacaccgggaacatcgatcgcagtgagctcaatcctaatgggtctggtcatggttggaagagcagcgttcgtctttccgttatcgtttctatctaacttagccaagaagaatcaaagcgagaaaatcaactttaacatgcaggttgtgatttggtggtctggtctcatgagaggtgctgtatctatggctcttgcatacaacaagtttacaagggccgggcacacagatgtacgcgggaatgcaatcatgatcacgagtacgataactgtctgtctttttagcacagtggtgtttggtatgctgaccaaaccactcataagctacctattaccgcaccagaacgccaccacgagcatgttatctgatgacaacaccccaaaatccatacatatccctttgttggaccaagactcgttcattgagccttcagggaaccacaatgtgcctcggcctgacagtatacgtggcttcttgacacggcccactcgaaccgtgcattactactggagacaatttgatgactccttcatgcgacccgtctttggaggtcgtggctttgtaccctttgttccaggttctccaactgagagaaaccctcctgatcttagtaaggct

SEQ ID NO: 4 - protein, 538 aa, Arabidopsis thaliana
MLDSLVSKLPSLSTSDHASVVALNLFVALLCACIVLGHLLEENRWMNESITALLIGLGTGVTILLISKGKSSHLLVFSEDLFFIYLLPPIIFNAGFQVKKKQFFRNFVTIMLFGAVGTIISCTIISLGVTQFFKKLDIGTFDLGDYLAIGAIFAATDSVCTLQVLNQDETPLLYSLVFGEGVVNDATSVVVFNAIQSFDLTHLNHEAAFHLLGNFLYLFLLSTLLGAATGLISAYVIKKLYFGRHSTDREVALMMLMAYLSYMLAELFDLSGILTVFFCGIVMSHYTWHNVTESSRITTKHTFATLSFLAETFIFLYVGMDALDIDKWRSVSDTPGTSIAVSSILMGLVMVGRAAFVFPLSFLSNLAKKNQSEKINFNMQVVIWWSGLMRGAVSMALAYNKFTRAGHTDVRGNAIMITSTITVCLFSTVVFGMLTKPLISYLLPHQNATTSMLSDDNTPKSIHIPLLDQDSFIEPSGNHNVPRPDSIRGFLTRPTRTVHYYWRQFDDSFMRPVFGGRGFVPFVPGSPTERNPPDLSKA

SEQ ID NO: 5 - DNA, 2232 bp, Medicago sativa
acgcggggaatccaacccattgtataacaacaactaccggagatatataatatctctctcctctaaatagaatatcgacagagtgactcaacaagattattaggagtgataatcttccacggcagctcaaaaacaaacaacatccgattcatcatcacgcgttgctcgagagatacttgtgttgatgagatcagaaggttcttaaaatggacagctcagaaacataaatatctgggattcattattactactggactttgaaatttggaaattcagcaataatctcaatttgttcttaaatctgcttttgaaatttgtggagggtggacgacatcatggctattgaaatgtcttctattgtttcaaaactatcaatgttatccacttccgatcatgcttctgttgtttctatgaacttgtttgtggcacttctgtgtgcttgtattgtccttggtcatcttctcgaggagaatcgatggatgaatgaatccatcactgcccttttgattggtatttgcactggtgtagtgattttgctgtttagtggtggaaaaagttcgcatattcttgttttcagtgaagatcttttctttatataccttctgccgcctattatattcaatgccgggtttcaagtaaagaaaaagcagttttttgtcaacttcatgactatcacatcatttggagctattggcacattaatatcttgtgtcattataaccacgggtgctacttttgcttttaagaggatggatattgggccactggaaatcggcgattatctagctattggagcaatatttgccgcaacagactctgtttgcacattgcaggtgctaaatcaggatgagacacctttattgtatagtcttgtatttggggaaggtgttgtgaatgatgctacctcagtggttcttttcaatgcaattcaaagctttgatcttaaccaactgaacccttcaattgcattgcatttcttgggcaacttcctgtatttgtttgtagcaagcacactccttggcgttgtgacaggtctgctcagtgcctatgttattaaaaagctgtacattggcaggcactccacagatcgtgaggttgctcttatgatgctaatggcatacctctcctatatgctggctgagttaacctatctgagtggcattcttaccgtattcttttgtggtattgttatgtctcattatacttggcataatgtgacgcagagttcaagaatcactaccaagcattcttttgctaccttgtcctttgttgctgagatctttatcttcctttatgttggtatggatgccctggacattgaaaaatggaagtttgttagtgatagtcctggaacatctatagctgcaagttcagtattgttgggtctaatacttcttggaagagcagcgtttgtttttcccttatccttcttatccaacttgactaaaaaatcacaacatcagaagatttccttcagacagcaagttatcatttggtgggctggtcttatgagaggtgctgtttcaatggcacttgcgtataatcagttcaccatgtcggggcatactcaactacgtagcaatgcaatcatgataaccagcaccatcactgttgtccttttcagcacagtggtgtttggtttgctgactaagccactcataaggcttctactacctcatcctaaaatcacaagcagcatgacaaccacagaatcgactactccaaaatcattcattgtcccacttctaggagattcccgagattctgaagctgatcttgaaggccatgaaattcaccgaccgaacagccttcgtgctttactatcaactccaactcacactgttcatcgattatggcgaaagtttgatgattcattcatgcgtcctgtttttggtggcagaggttttgttcctgtagaacctggctcaccaagtgaacgcaatggtaatcaatggggttgagaaaagaagccatgaaatgtgtaatatgtgttgtatactacgtatgatttgtgaaaagtcatgcaacgtgtgtataatgtatttattgcataagaacctagtagtgaaatttttctttaaaaaaaaacctcgtagtgaaattttgttgagctgtttgagtagctagtatgagatggcttgccatctctctgtctattatgtaaactacaatattttttagattctctgagccattacatgtttgtgtatgtgtccaaaaaaaaaaaaaaaa

SEQ ID NO: 6 - protein, 541 aa, Medicago sativa
MAIEMSSIVSKLSMLSTSDHASVVSMNLFVALLCACIVLGHLLEENRWMNESITALLIGICTGVVILLFSGGKSSHILVFSEDLFFIYLLPPIIFNAGFQVKKKQFFVNFMTITSFGAIGTLISCVIITTGATFAFKRMDIGPLEIGDYLAIGAIFAATDSVCTLQVLNQDETPLLYSLVFGEGVVNDATSVVLFNAIQSFDLNQLNPSIALHFLGNFLYLFVASTLLGVVTGLLSAYVIKKLYIGRHSTDREVALMMLMAYLSYMLAELTYLSGILTVFFCGIVMSHYTWHNVTQSSRITTKHSFATLSFVAEIFIFLYVGMDALDIEKWKFVSDSPGTSIAASSVLLGLILLGRAAFVFPLSFLSNLTKKSQHQKISFRQQVIIWWAGLMRGAVSMALAYNQFTMSGHTQLRSNAIMITSTITVVLFSTVVFGLLTKPLIRLLLPHPKITSSMTTTESTTPKSFIVPLLGDSRDSEADLEGHEIHRPNSLRALLSTPTHTVHRLWRKFDDSFMRPVFGGRGFVPVEPGSPSERNGNQWG

SEQ ID NO: 7 - DNA, 2263 bp, Suaeda maritima subsp. salsa
tttcacaaagattattggacttcagaagtttgattttgtggagctagaaagggtttcacatacattggacattaatttacttgaatatatatatatttgttgtgggtcttggattcgggtgcacaaagaaataggtgaacaatgttgtcacagttgagctctttttttgcaagtaagatggacatggtttcgacgtctgatcatgcttccgttgtttcgatgaatttgtttgtggcactgttacgtggctgcattgtaattggtcatcttctcgaagagaatcgctggatgaatgaatccattacagctttgctaataggtttatctactgggattataatcctgctaattagtggaggaaagagttcgcatttgttggtcttcagtgaagatcttttctttatatacctccttccaccgattatattcaatgcggggtttcaggtgaaaaagaagcaatttttccgcaacttcattactattattttgtttggagccgttggtacattggtatcattcataatcatatctcttggttcaatagctatatttcaaaagatggatattggttcgctggagttaggggatcttcttgcaattggtgcaatattcgctgcaactgattcagtttgcacattgcaagtgcttaatcaagatgagactccacttctttatagtctcgtgtttggtgaaggtgtcgtcaatgatgctacatcagtggtgttgttcaatgcaattcaaaactttgacctcacgcacattgaccacagaattgccttccaatttggtggcaactttctatatttattttttgcaagcactctgcttggagcagtgactggcttgctaagcgcttatgtcatcaaaaagttgtactttggaaggcattcaactgaccgtgaggtagccttaatgatgcttatggcttatctatcgtacatgcttgctgaactcttctatctgagcggaattcttacagtattcttctgtgggattgtcatgtcccattatacatggcacaatgtgacggagagctccagagtaaccaccaagcatgcttttgcaacactctcttttgtagctgagatcttcatctttctatatgttggtatggatgcactggatattgagaagtggagatttgtgagcgatagtcctggaacatctgttgctgtgagttccatactgcttggtcttcacatggttgggcgagctgcttttgtttttcccttcgcctttttaatgaacttgtccaagaaatcaaatagtgagaaggtcaccttcaatcagcagatagtcatttggtgggctggtctcatgaaaagtgctgtctccgtggcacttgcttataatcagttttcaaggtcaggacacacacagctgaggggaaatgcaatcatgattacaagcaccataaccgttgtccttttcagtacgatggtatttgggttgctgacaaagcctcttatactctttatgttgcctcaaccgaaacatttcactagtgcaagcaccgtgtcagatttggggagtccaaagtcattctccttgcctcttcttgaagatagacaagattctgaagctgatttgggcaacgatgatgaagaagcctacccccgtgggactatagctcgacctactagtcttcgtatgctactaaatgcaccaactcacactgtccatcattattggcgcagattcgatgattatttcatgcggcctgtatttggtggccggggttttgtaccttttgtcccaggttcacccaccgaacagagcatcactaattttgtcacagagaacataagttagcgataattgaggcagttggtgcagaaactaataacttacagccctacaggcaatctacaaagacaaaaaatgcccttacccaagaacgaacagcccggtgtttggtctcgtgggcttgatgttaagactgtgctgtacttctgttaatagagagtaagttacagaaaccaccgatttaaacatatctgtaattttttacagcatggatattcgatgcattctttaatctggctgtagctagaatactctagcatgttttgtagtttcagtcttaccatttaggttttctcctacataacctcaataagctgtttagtgtgcttactgcttactttagagcaaactgcaactgtgaaaattgcttacgtcagcggcacctgtgtaatttatcatttttataatgatggagcatgatcatttgcaatcaaatttacaatactgtgattaaaaa

SEQ ID NO: 8 - protein, 556 aa, Suaeda maritima subsp. salsa
MLSQLSSFFASKMDMVSTSDHASVVSMNLFVALLRGCIVIGHLLEENRWMNESITALLIGLSTGIIILLISGGKSSHLLVFSEDLFFIYLLPPIIFNAGFQVKKKQFFRNFITIILFGAVGTLVSFIIISLGSIAIFQKMDIGSLELGDLLAIGAIFAATDSVCTLQVLNQDETPLLYSLVFGEGVVNDATSVVLFNAIQNFDLTHIDHRIAFQFGGNFLYLFFASTLLGAVTGLLSAYVIKKLYFGRHSTDREVALMMLMAYLSYMLAELFYLSGILTVFFCGIVMSHYTWHNVTESSRVTTKHAFATLSFVAEIFIFLYVGMDALDIEKWRFVSDSPGTSVAVSSILLGLHMVGRAAFVFPFAFLMNLSKKSNSEKVTFNQQIVIWWAGLMKSAVSVALAYNQFSRSGHTQLRGNAIMITSTITVVLFSTMVFGLLTKPLILFMLPQPKHFTSASTVSDLGSPKSFSLPLLEDRQDSEADLGNDDEEAYPRGTIARPTSLRMLLNAPTHTVHHYWRRFDDYFMRPVFGGRGFVPFVPGSPTEQSITNFVTENIS

SEQ ID NO: 9 - DNA, 1623 bp, Zea mays
atggggcttggagtagtggcggagctagtccgccttggcgtcctttcctccacctcagatcacgcctccgtggttagcatcaatctctttgtcgccttgctctgcgcctgtatcgtcctgggccatcttcttgaagagaataggtgggtgaacgagtccaccgcgctgattgtcgggctcggcaccggtaccgtcatcctcatgattagccggggggtggttattcacgtcctagtcttctccgaggacctcttcttcttctatcttttgccgccgatcattttcaatgcagggttccaagtgaagaagaaacagttctttcgaaacttcattactattacactgtttggtgcagttggcaccttgatctcttttactgtaatatcccttggcgctctaggactaatatcaaggcttaatatcggcgcacttgaactgggagactatcttgcacttggggcaatattctcggccacagactcggtttgcaccttgcaggtgttaagccaagatgagacaccattcttgtacagtcttgtattcggtgaaggcgtggtcaacgatgccacttccgtagtggtgttcaatgcactccaaaactttgatataactcacatcgatgcggaggttgtcttccatctattaggaaacttcttctacctcttccttctatcaactgtgttgggagtggccacaggacttatctcagcgttagtgattaaaaagctatactttggacggcactctactgacagggaggtggctcttatgatgcttatggcgtatctctcctacatgttggcggaactcttcgcgctgagcgggatcttgacggtattctttgggtgcattgttatgagccactatacatggcacaacgtgacagagtccagcagaatcacgactaagcatgcgtttgccacgctcagcttcctagccgaaaccttcctctttctgtacgtgggtatggatgctctcgacattgacaagtggaggtccgtgagtgacaccccaggtaagtctctggccataagctcgattttgatgggactcgtgatggttggccgggctgccttcgtattccctctctccttcctctccaatttagcgaaaaaaacggagcacgaaaaaatcagctggaagcagcaggtggtcatttggtgggcgggtctcatgcgaggcgccgtttcgatggccctagcgtacaagaagtttacccgcgcagggcatactcaggtccgcgggaacgcgatcatgattaccagcacgattatcgtcgtgttgttttcgacaatggtgttcggcctgctcacgaagcccttaattaacttgctaataccgcaccgtaacgccacatcgatgttgagcgatgactcaagcccaaagtccttgcatagccctctgctaacctctcaactcggtagcgacttagaggagccgaccaacatcccgcggccgagctccataagaggcgagttcctcaccatgactaggaccgtgcaccgatactggcgcaagttcgacgacgccttcatgaggcccatgttcggaggccgcggtttcgtacctttcgtgccaggcagcccgaccgagcgtaatccgccggatctttccaaggcttaa

SEQ ID NO: 10 - protein, 540 aa, Zea mays
MGLGVVAELVRLGVLSSTSDHASVVSINLFVALLCACIVLGHLLEENRWVNESTALIVGLGTGTVILMISRGVVIHVLVFSEDLFFFYLLPPIIFNAGFQVKKKQFFRNFITITLFGAVGTLISFTVISLGALGLISRLNIGALELGDYLALGAIFSATDSVCTLQVLSQDETPFLYSLVFGEGVVNDATSVVVFNALQNFDITHIDAEVVFHLLGNFFYLFLLSTVLGVATGLISALVIKKLYFGRHSTDREVALMMLMAYLSYMLAELFALSGILTVFFGCIVMSHYTWHNVTESSRITTKHAFATLSFLAETFLFLYVGMDALDIDKWRSVSDTPGKSLAISSILMGLVMVGRAAFVFPLSFLSNLAKKTEHEKISWKQQVVIWWAGLMRGAVSMALAYKKFTRAGHTQVRGNAIMITSTIIVVLFSTMVFGLLTKPLINLLIPHRNATSMLSDDSSPKSLHSPLLTSQLGSDLEEPTNIPRPSSIRGEFLTMTRTVHRYWRKFDDAFMRPMFGGRGFVPFVPGSPTERNPPDLSKA

SEQ ID NO: 11 - DNA, 1623 bp, Zea mays
atgggccttggtgttgatgcggagacggtcaggctcggagtccttagctcgacctcggatcatgccagcgttgtcagtaacaacttcttcgtagcacttctttgcgcctgtatcgtcctcgggcatctcttggaggagaaccgaatggttaatgagtctattacagcactgctggtggggctgggcactgggaccgtgattctgatgattagtcggggcgtgagtattcacgttctcgtcttttcagaggacctgttctttatctatctgttacctccgattatcttcaatgccgggtttcaagtaaagaaaaagcaattcttccgcaactttataacgattattttgtttggtgctattgggactctgatttcctttgtaataatctctcttggtgctatggggttgttcaagaaacttgatgttggtccactcgagcttggggactatcttgcaattggtgctattttctcggcaacagattctgtttgcaccttacaggtgcttaaccaggatgaaacacccctactctacagtctcgtattcggcgagggcgttgttaatgatgctacctcaatcgttgtgttcaacgcgctccaaaacttcgacatcacccacatcaatgccgaggtggtatttcacctccttggcaacttcttgtacctcttcctattgagcaccgtgctcggcgtggcgaccggtctcatctccgcgctggtcattaagaagatctacttcggacgccactcgactgatcgggaagtggccttaatgatgctgatggcatatctaagctacatgctggcagagctttttgccctgtccggaatcctcactgtgtttttcggctgcatcgtcatgagccattatacgtggcacaacgtcacggagtctagccgaattactacgaagcacgcctttgccaccctgtctttcctcgctgagactttcatatttctctacgttgggatggatgcgctagacattgagaagtggcggtccgtttcggacaccccgggcaaatcgatagccatatcctccatactcatggggcttgtcatgcttggacgcgcggctttcgtgttcccgctaagtttcttgtcaaatctggcgaagaagaatgagcacgaaaagatctcctggaagcagcaagttgtgatctggtggagcggtttgatgaggggtgctgtctctatggccctagcttataacaagtttaccagagccggccatacggaggtgagaggtaacgaaatcatgattactagcaccattaccgtcgtgctattctccacagtggtgttcggtctcctgactaaaccactgatcaggctccttatgccccaccgccatctgaccatgctctccgacgacagcaccccgaagtcattgcactcacctttgctgacatcccagctcggaagctccatcgaagagccgacgcagataccacgccctacaaatattcgtggcgaattcacaactatgacgagaacggtgcataggtactggagaaaatttgatgacaaattcatgcgcccaatgtttggcggcaggggcttcgtacccttcgtccctggttcaccaacggagaggaatccccacgatctttcgaagccctaa

SEQ ID NO: 12 - protein, 540 aa, Zea mays
MGLGVDAETVRLGVLSSTSDHASVVSNNFFVALLCACIVLGHLLEENRMVNESITALLVGLGTGTVILMISRGVSIHVLVFSEDLFFIYLLPPIIFNAGFQVKKKQFFRNFITIILFGAIGTLISFVIISLGAMGLFKKLDVGPLELGDYLAIGAIFSATDSVCTLQVLNQDETPLLYSLVFGEGVVNDATSIVVFNALQNFDITHINAEVVFHLLGNFLYLFLLSTVLGVATGLISALVIKKIYFGRHSTDREVALMMLMAYLSYMLAELFALSGILTVFFGCIVMSHYTWHNVTESSRITTKHAFATLSFLAETFIFLYVGMDALDIEKWRSVSDTPGKSIAISSILMGLVMLGRAAFVFPLSFLSNLAKKNEHEKISWKQQVVIWWSGLMRGAVSMALAYNKFTRAGHTEVRGNEIMITSTITVVLFSTVVFGLLTKPLIRLLMPHRHLTMLSDDSTPKSLHSPLLTSQLGSSIEEPTQIPRPTNIRGEFTTMTRTVHRYWRKFDDKFMRPMFGGRGFVPFVPGSPTERNPHDLSKP

SEQ ID NO: 13 - DNA, 1620 bp, Zea mays
atgtcaataggactgacggccgagaccgtgactaacaagctagccagcgccgagcacccccaagtcgtccctaattctgtgttcattgcgctcctctgtctgtgcctggtgataggccacctccttgaggagaacagatgggtcaatgaatcaataacagccattctcgtgggcgctgcgactgggaccgtcatcctgctcatctcgaaaggaaaatcgagccacatacttgtgttcgatgaggaattgtttttcatctatctactgccgccaattattttcaatgccgggtttcaagtaaagaaaaagcaattcttccgcaactttataacgattattttgtttggtgctattgggactctgatttcctttgtaataatctctcttggtgctatggggttgttcaagaaacttgatgttggtccactcgagcttggggactatcttgcaattggtgctattttctcggcaacagattctgtttgcaccttacaggtgcttaaccaggatgaaacacccctactctacagtctggtattcggtgagggggtcgtgaacgacgctacaagtgttgtgctgtttaatgcagtgcaaaagatcgacttcgaacaccttaccggagaggtggcgctccaggttttcggcaacttcctctatctgttctcaacctcaacggtcctgggcatagccactgggctcattaccgccttcgtcctcaagacactctacttcggccgtcatagtactacccgtgagttggccattatggtcctgatggcctacttgtccttcatgcttgctgagttgttcagtctcagtggtatcattactgtttttttctgcggcgtgctcatgtcccatgttacctggcacaatgttactgagtcgtccagaattacctctcgccatgtgttcgctatgctaagcttcattgccgaaacgtttttgtttctgtacgtggggacggacgcgcttgacttcacaaagtggaagacgtcttcgttatcctttgggaagtccctaggggtatccagcgtgctcctggggttggttctagtcggtcgggcggcattcgttttccccctctcgttcctgagcaaccttagtaagaaacaccctggggaaaaaatcacgatcaggcagcaggttgtaatttggtgggcaggacttatgaggggcgccgtcagcatcgctttggcgttcaacaaatttacaagggccggtcacactcaggtaagaggaaacgcaatcatgatcactagcaccatcatcgtggtgcttttctctacagtcgttttcggcctcctcaccaaaccgttaatcaaccttctcataccccatcgcaatgcaacctccatgttgtctgacgactccagccctaagtctctacacagcccacttttaacctcccaactgataagctcaatcgaggagcccacgcaaatcccgcggccgacaaatatacggggtgagttcatgaccatgacgcgaaccgtgcatcgctattggcgcaagtttgatgacaagttcatgaggcctatgttcggaggcaggggttttgtcccgtttgtcccagggtcgcctaccgaaagaagctcacccgatctatccaaggcatga

SEQ ID NO: 14 - protein, 539 aa, Zea mays
MSIGLTAETVTNKLASAEHPQVVPNSVFIALLCLCLVIGHLLEENRWVNESITAILVGAATGTVILLISKGKSSHILVFDEELFFIYLLPPIIFNAGFQVKKKQFFRNFITIILFGAIGTLISFVIISLGAMGLFKKLDVGPLELGDYLAIGAIFSATDSVCTLQVLNQDETPLLYSLVFGEGVVNDATSVVLFNAVQKIDFEHLTGEVALQVFGNFLYLFSTSTVLGIATGLITAFVLKTLYFGRHSTTRELAIMVLMAYLSFMLAELFSLSGIITVFFCGVLMSHVTWHNVTESSRITSRHVFAMLSFIAETFLFLYVGTDALDFTKWKTSSLSFGKSLGVSSVLLGLVLVGRAAFVFPLSFLSNLSKKHPGEKITIRQQVVIWWAGLMRGAVSIALAFNKFTRAGHTQVRGNAIMITSTIIVVLFSTVVFGLLTKPLINLLIPHRNATSMLSDDSSPKSLHSPLLTSQLISSIEEPTQIPRPTNIRGEFMTMTRTVHRYWRKFDDKFMRPMFGGRGFVPFVPGSPTERSSPDLSKA

SEQ ID NO: 15 - DNA, 1617 bp, Zea mays
atggggtatcaggtcgtcgccgcgcagctgaagctggcttcctcagctgaccacgcaagcgtggttatcatcacgctcttcgtggccctcctctgcgcttgcatagtgttgggccatcttcttgaagagaatcgctggctaaacgaatcaattacagcattgataatcgggctcggaacgggggttgtgattctattgatcagccgaggtaagaacagccgcctgcttgtgttctcggaggacctcttcttcatctatctattgccgcccattattttcaatgccgggttccaggtgaagaagaaacagttcttccggaatttcatgacaatcacactattcggtgctgttggcacaatgatatccttcttcacaatctctctcggcgcaatagcgacattcagcagaatgagcattgggacgctagatgtcggggattttctcgctattggagctatcttttctgcaacggattctgtgtgcacgctgcaggtcctccatcaggatgagacgccctttctgtacagtctggtattcggggagggcgtagtgaacgatgccacaagtgttgtactcttcaacgcagttcagaagatccagttcacccacataaatgcatggacagctctccagctgatcggtaactttctttacctcttctccacgagtacactgctcggtatcgggaccggcttgatcacagcgtttgtcctgaagaagttgtatttcggcaggcactccactacccgggagcttgcgatcatgatcttaatggcctacctgtcatacatgcttgccgagttgtttagtctgtccgggctcctcacggtctttttctgtggcgtgctaatgtctcatgtcacatggcataatgttacggagtccagcaggacaaccagccgtcacgtgttcgcgacgctctcgttcatatctgagactttcatattcctgtatgtgggcatggacgcactcgatttcgagaagtggaagacctcatcattaagcttcggtgggaccctgggagttagtggagtactcatggggctggtcatgctaggcagagctgcttttgtctttcctctctcctttctctccaacctcgccaagaaacaccaaagtgagaaaatttcttttaggatgcaggttgtgatttggtgggcgggtctaatgcgcggcgcggtttccatggccttggcgttgaacaaattcactcggagcggccacacccagctacatggcaatgctatcatgataacttcaaccattaccgtggtgctgttctctacgatggtctttggcatgattacaaagccactgatcaggctgcttttgcctgcgtctggacatccgagagaattatcggaaccgtcgtcacccaagagcttccatagtcctcttcttacctcgcaacagggatctgacctggagtcgacaaccaatatagtccgtccctcctcacttagggggctcctcactaaaccaactcacacggtgcactactactggcggaagttcgatgacgcacttatgagaccggtgttcgggggacgtggtttcgtgccatttgttcccggcagcccaaccgagcgaaatccacccgatctgtccaaagcctga

SEQ ID NO: 16 - protein, 538 aa, Zea mays
MGYQVVAAQLKLASSADHASVVIITLFVALLCACIVLGHLLEENRWLNESITALIIGLGTGVVILLISRGKNSRLLVFSEDLFFIYLLPPIIFNAGFQVKKKQFFRNFMTITLFGAVGTMISFFTISLGAIATFSRMSIGTLDVGDFLAIGAIFSATDSVCTLQVLHQDETPFLYSLVFGEGVVNDATSVVLFNAVQKIQFTHINAWTALQLIGNFLYLFSTSTLLGIGTGLITAFVLKKLYFGRHSTTRELAIMILMAYLSYMLAELFSLSGLLTVFFCGVLMSHVTWHNVTESSRTTSRHVFATLSFISETFIFLYVGMDALDFEKWKTSSLSFGGTLGVSGVLMGLVMLGRAAFVFPLSFLSNLAKKHQSEKISFRMQVVIWWAGLMRGAVSMALALNKFTRSGHTQLHGNAIMITSTITVVLFSTMVFGMITKPLIRLLLPASGHPRELSEPSSPKSFHSPLLTSQQGSDLESTTNIVRPSSLRGLLTKPTHTVHYYWRKFDDALMRPVFGGRGFVPFVPGSPTERNPPDLSKA

SEQ ID NO: 17 - DNA, 2564 bp, Hordeum vulgare
aacggaaccttctccagataccccgcccgcgcgaaaagaatagaggagaatcccgacctccccgcccgcgcggctgcgcatctgccccccctccttctccctcctcgctccccaccccgggtttcccgtgccattctttccctccccaccccggccccgggcacgaagcagcggcggagacggggccaggaggaggaggagctcggctgttcttcgtctccccgtcgattcgtctccggattagcgccgccggccgttccccgagggctccgtccgggttgattcgatctgattgaaaaagcccgcgtctttccccgagggcgcgcgctcgctcgccggagctagctgtgtctcgttcggccgggctcaaggaagaagagtaacgggcgggatggcgttcgaagtggtggcggcgcagttggcgcggctgagcgacgcgctggccacctcggaccacgcctccgtggtctccatcaacctcttcgtcgcgctgctctgcgcctgcatcgtcctcggccacctcctcgaggagaaccgctggctcaacgagtccatcaccgccctcatcatcgggctgtgcaccggcgtggtgatcctgatgaccaccaaggggaagagctcgcacgtgctcgtcttcagcgaggacctcttcttcatatacctcctccctcccatcatcttcaacgccggtttccaggtgaagaagaagcagttcttccggaatttcatgacaatcacattattcggcgctgtcgggacgatgatttcattcttcacaatctctcttgctgccattgcgatattcagcaagatgaacattgggacactggatgtatcagattttctcgcaattggagccatcttttccgcgacagattctgtctgcactttacaggttctcaatcaggacgagacgccctttctgtacagtctagttttcggggaaggtgttgtgaacgatgccacatcagtcgtgcttttcaacgcgctccagaacttcgatcctaaccaaatcgatgcaatcgtcattctgaagttcttgggaaacttctgctacttattcgtgtcaagcaccttccttggagtattttctggattgctcagtgcatacataatcaagaagttatacataggaaggcattctactgaccgtgaggttgcgcttatgatgctcatggcctacctctcatatatgctagctgagctgcttgatttgagtggcatcctcaccgtgttcttctgtggtattgtgatgtcgcattatacttggcataatgtgacagagagctcaagagttacaacaaagcatgcttttgcaaccttgtccttcattgctgagacctttctcttcctttatgttgggatggatgcactggatatcgagaagtggaaatttgctagtgacagccctggcaaatccatcggaataagctcaattttgctaggattagttctggttggaagagctgcttttgtcttcccgctttcattcttatccaacctgacaaagaagacggagctcgaaaaaataagctggaggcagcaaatcgtaatatggtgggctgggctgatgagaggagctgtgtcgatcgctcttgcttacaataagtttacaagatctggccacacacagctacacggcaacgcgataatgatcaccagcaccatcactgtcgttctgtttagcactatgctgtttggcatattgacaaagcctctgatccggttcctgctgcccgcgtcgagcaatggcgacccctcggagccctcgtcaccgaagtccctgcactctcctctcctcacaagcatgctaggctcggacatggaggcgcctctccccatcgtcaggccctccagcctccggatgctcatcaccaagccgacccacaccatccactactactggcgcaagttcgacgacgcgctgatgcgtcctatgttcggcgggcgcgggttcgtgccctactcccctggatcacccaccgatccaaacgtaatcgtggcatgaacgttgtggagagaagagaaaagccattacagcttcaggagacactctgaactgttgtaactggaagagaaggaggtgctacagcttcggaagaaggcgaagtctccattactattatagtgtttggctgactcggagggccgaagaaggcgcccctctgacgatggttcagatgaacggttggttgcggcaccaacaggaagatgaaccctagtaacggtgatgcgagtaccatcgccttatcggttacgacaagcctgtacatttttgtatgtagattaacaagccaattgtaccctatgagatgagatctcctctggcaggcaggcaggccatttccttgctccttggctaggagtctctggcctcctgcatatctaccagtgcttattaatctcctcccccactttctagtggattggtgtaatggtgtgtactttaccaagttgtgtgagatgagtgatgatcttgtggcctggttgctacaaagaactcatctcaaagttatctatctattttctatattgaattgaactgaacttgtgtcttgaaccac

SEQ ID NO: 18 - protein, 538 aa, Hordeum vulgare
MAFEVVAAQLARLSDALATSDHASVVSINLFVALLCACIVLGHLLEENRWLNESITALIIGLCTGVVILMTTKGKSSHVLVFSEDLFFIYLLPPIIFNAGFQVKKKQFFRNFMTITLFGAVGTMISFFTISLAAIAIFSKMNIGTLDVSDFLAIGAIFSATDSVCTLQVLNQDETPFLYSLVFGEGVVNDATSVVLFNALQNFDPNQIDAIVILKFLGNFCYLFVSSTFLGVFSGLLSAYIIKKLYIGRHSTDREVALMMLMAYLSYMLAELLDLSGILTVFFCGIVMSHYTWHNVTESSRVTTKHAFATLSFIAETFLFLYVGMDALDIEKWKFASDSPGKSIGISSILLGLVLVGRAAFVFPLSFLSNLTKKTELEKISWRQQIVIWWAGLMRGAVSIALAYNKFTRSGHTQLHGNAIMITSTITVVLFSTMLFGILTKPLIRFLLPASSNGDPSEPSSPKSLHSPLLTSMLGSDMEAPLPIVRPSSLRMLITKPTHTIHYYWRKFDDALMRPMFGGRGFVPYSPGSPTDPNVIVA

SEQ ID NO: 19 - DNA, 2422 bp, Triticum aestivum
atggggtaccaagtggtggcggcgcagctggcgcggctgagcggcgcgctgggcacctcggaccacgcctccgtggtctccatcaccctcttcgtcgcgctgctctgcgcctgcatcgtcctcggccacctgctcgaggagaaccgctggctcaacgagtccatcaccgccctcatcatcgggctgtgcaccggcgtggtgatcctgatgaccaccaaggggaagagctcgcacgtgctcgtcttcagcgaggacctcttcttcatctacctcctgcctcccatcatcttcaacgccggtttccaggtgaagaagaagcagttcttccggaatttcatggcaatcacactatttggtgccgttgggacgatgatgtcgtttttcacaatatctcttgctgccattgcgatattcagcaggatgaacattgggacactggatgtatcagattttcttgcaattggagctatcttttccgcgacagattctgtctgcactctacaggttctcaatcaggacgagacgccctttttgtacagtctagtgttcggggaaggtgttgtgaacgatgccacatcggtcgtgcttttcaacgcgctccagaacttcgatcctaaccagatcgacgcgatcgtcattcttaagttcttggggaacttctgctacttattcgtgtcaagcaccttccttggagtgtttactggattgcttagtgcatacgtcatcaagaagttatacataggaaggcattctactgaccgtgaggtcgcacttgtgatgctcatggcctacctctcatatatgctagctgagctgctagatttgagtggtatcctcactgtattcttctgtggtattgtgatgtcacattacacctggcacaacgtgacagagagctcaagagttacaacaaagcatgcatttgcaaccttgtccttcatcgctgagacttttctcttcctttatgttgggatggatgcactggatattgagaagtggaaatttgctagtgacagccccggcaaatccattggaataagctcaattttgctcgggttggttctggttggaagagctgctttcgtcttcccgctctcgttcttatccaacctgacaaagaagacggagctcgaaaaaataagctggaggcagcaaatcgtaatatggtgggctgggctgatgagaggagctgtgtcgatcgctcttgcttacaataagtttacaagatctggtcacacacagctgcacggcaacgcgataatgatcaccagcaccatcactgtcgttctgtttagcactatgttgtttggcattttgacaaagcctctgatccggttcctactgcccgcgtcgagcaatggcgccgcctcagatcccgcgtcaccgaagtccctgcactctcctctcctcacaagccagctaggctcggacctggaggcgcctctccccatcgtgaggccctccagcctccggatgctcatcaccaagccgacccacaccatccactactactggcgcaagtttgacgacgcgctgatgcgcccgatgttcggagggcgcgggttcgtgccctactccccaggatcacccaccgatccgaacgtactcgtggaatgaacgtcgcgaagaagcaacggagaagccattacagcttcaggagacactctgaactgtaacaggaagggaaggaagtgtcacagcttcagaagaacgcgaagtctccggtaatattatagcgtttggcagactcggaaggctgaagaaggcggccctccgatgatggttcagatgaacggttggttgcggcaccgacaggaagatgaaccctagtaacggtgatgcgagtatcatcatcgccttatcggttacgacaaagcctgtacagttttgtatgtagattaacaagccaattgtatcctatgagatctccgttggcaggcaggcgtctgacctcctgcatctgcgacgaccgcggcgtggccaaggccgggtgcgggcggtcgtacgcgccgttcccgcccgggtgcatgttccacagcgagggcgggctcaagagcttcgagcaccccatgaaccgccttaaggcgctccccagggtggacagcgagggcgtcatgtgcggcgccaacttcaaggtcgacgccttcaccaagatcaactccatgccccgcgtcggcagcgccaccaactgggccgccgcctgggacgacgccgccatctgatcctcgccggcgccggcgttgctctccgtcgtggcctcgtcgggcttgggcttattgcattttacttgtttttctttccttggcaatgtacattctgatctgatctgatctgagccgtgtgtgggcgtgggcgcgctggcacgtacggctgtttgcttgtacgatggaggaataagactttgcttccagtccaaaaaaaa

SEQ ID NO: 20 - protein, 538 aa, Triticum aestivum
MGYQVVAAQLARLSGALGTSDHASVVSITLFVALLCACIVLGHLLEENRWLNESITALIIGLCTGVVILMTTKGKSSHVLVFSEDLFFIYLLPPIIFNAGFQVKKKQFFRNFMAITLFGAVGTMMSFFTISLAAIAIFSRMNIGTLDVSDFLAIGAIFSATDSVCTLQVLNQDETPFLYSLVFGEGVVNDATSVVLFNALQNFDPNQIDAIVILKFLGNFCYLFVSSTFLGVFTGLLSAYVIKKLYIGRHSTDREVALVMLMAYLSYMLAELLDLSGILTVFFCGIVMSHYTWHNVTESSRVTTKHAFATLSFIAETFLFLYVGMDALDIEKWKFASDSPGKSIGISSILLGLVLVGRAAFVFPLSFLSNLTKKTELEKISWRQQIVIWWAGLMRGAVSIALAYNKFTRSGHTQLHGNAIMITSTITVVLFSTMLFGILTKPLIRFLLPASSNGAASDPASPKSLHSPLLTSQLGSDLEAPLPIVRPSSLRMLITKPTHTIHYYWRKFDDALMRPMFGGRGFVPYSPGSPTDPNVLVE

SEQ ID NO: 21 - DNA, 1726 bp, Oryza sativa
ggtggccatctcgcttgaatctgcagggtgagctgaggaggatccactgaggtggcggcggtcgagatggggctggatttgggagctctcgttctcaaatccggcgggctgttggtgtcggactacgactcgatcgtcgcgatcaacatcttcgtggcgctgctgtgcagctgcattgtgatcgggcacctgctggaagggaaccggtgggtcaatgaatccatcaccgcgcttgtcatggggctgatcactggaggtgtgattctgctcgtcagtggtgggaagaactcgcacattcttgtgttcagtgaggacctcttcttcatttatttgcttccaccgatcatctttaatgctgggtttcaagtaaagaaaaaacaattcttccgcaattttatgacaattattttatttggtgctgtggggacattgatatcctttgtgataatctctctaggtgccatgacattgttcaaaaaacttgatgttggtccactccagcttggggactatcttgcaattggggctatcttctcagcaacagattctgtttgcaccttacaggtgcttaaccaagacgaaacacccctactctatagtctggtttttggtgaaggggttgtcaatgatgctacatctgttgtgctctttaatgcaattgaagacattgatattgctaattttgatagccttgttctactagcgttcataggaaattttctctacctattcttcaccagtacccttcttggagtagttgctgggttgcttagtgcctatattattaagaaactatgttttgccagacactcaactgacagagaagttgctatcatgatactcatggcgtacctttcatatatgctgtcgatgctgctagatctgagtggcattctcactgtgttcttctctggaatagtaatgtcacattacacttggcataatgtgacagaaagctctaggattactaccaagcacacttttgctactttatctttcattgctgaaatttttctatttctctatgttgggatggatgcactggacattgaaaaatggaaattagctagcagcagtcctaaaaaaccaattgctttaagtgcaactatattgggcttggttatggttggaagagcagcatttgtattccctttgtctttcttatccaatctaagtaaaaaagagacacgcccaaagatctccttcaagcagcaagtaatcatatggtgggcaggtctcatgagaggagcagtatcaatagcacttgcctatcacaagttcaccgcatctggtcatactgaattgcgaatcaatgctatcatgatcaccagcacagtcattgttgttctgttcagcacaatggtttttggtttttttaccaagcctctcctcaatctcctcatcccaccaaggcctgacatagcagctgatctctcaagccagtcaatcatagacccacttcttggaagcctgctggggtctgacttcgatgtaggccagccctcccctcagaacaaccttcagcttcttctcaccattcagactcgctccgttcatcgcgtgtggcgcaagtttgatgatagattcatgcgcccgatgttcgggggccgaggcttcgttcctttcgtgcctggttcgccagtggagcggagcatccatggatctcaactgggcactgtgactgaggctgaacatagctgagtttgaggttcagaaggtgcaagca

SEQ ID NO: 22 - protein, 544 aa, Oryza sativa
MGLDLGALVLKSGGLLVSDYDSIVAINIFVALLCSCIVIGHLLEGNRWVNESITALVMGLITGGVILLVSGGKNSHILVFSEDLFFIYLLPPIIFNAGFQVKKKQFFRNFMTIILFGAVGTLISFVIISLGAMTLFKKLDVGPLQLGDYLAIGAIFSATDSVCTLQVLNQDETPLLYSLVFGEGVVNDATSVVLFNAIEDIDIANFDSLVLLAFIGNFLYLFFTSTLLGVVAGLLSAYIIKKLCFARHSTDREVAIMILMAYLSYMLSMLLDLSGILTVFFSGIVMSHYTWHNVTESSRITTKHTFATLSFIAEIFLFLYVGMDALDIEKWKLASSSPKKPIALSATILGLVMVGRAAFVFPLSFLSNLSKKETRPKISFKQQVIIWWAGLMRGAVSIALAYHKFTASGHTELRINAIMITSTVIVVLFSTMVFGFFTKPLLNLLIPPRPDIAADLSSQSIIDPLLGSLLGSDFDVGQPSPQNNLQLLLTIQTRSVHRVWRKFDDRFMRPMFGGRGFVPFVPGSPVERSIHGSQLGTVTEAEHS

SEQ ID NO: 23 - DNA, 1902 bp, Saccharomyces cerevisiae
atgctatccaaggtattgctgaatatagctttcaaggtgctgttaaccaccgccaagagagcagttgatcctgacgatgatgatgaacttctaccttccccggatctcccgggtagcgatgaccctattgcaggtgatcctgatgtagacttaaaccctgttacagaagaaatgttctcttcatgggcattgttcattatgttgctcctattgatctctgcattgtggtctagttactatttaactcagaaacgaattagggcagtgcatgaaactgtgctttctattttttatggtatggttattggcttgataataaggatgtcccccgggcattatattcaagatacggttacttttaattcatcctacttttttaatgttctattgccgccaattattttaaatagtgggtacgagttgaatcaagtgaactttttcaataatatgttatctatcttaattttcgccataccgggcaccttcatatctgctgtggttattggaatcatattgtatatctggacctttttaggactagagagtattgacatttcattcgcagatgcaatgtctgttggtgctacattatctgctaccgaccctgttacaattctttcaattttcaatgcgtataaagtggatcctaagctatataccatcatttttggagaatcactgttaaatgatgccatctctattgttatgtttgaaacctgtcaaaaatttcatggtcaacctgcaacattttcgtcggtttttgaaggggcaggcctctttttgatgactttctccgtttcgttgttgataggcgttcttataggaattcttgttgctcttctgttgaaacacactcacataaggcgctatcctcaaattgagagttgtttgatcttgttgattgcttatgaatcctattttttctccaacggttgccatatgtccggtatcgtctccttgttattttgcggaattactttaaaacattacgcctattataacatgtcaagaagatcacagatcaccattaagtatattttccaactattggcaagattatcagagaatttcatctttatctatctaggtttagaactttttactgaagtagaactagtctataagccactgctaattattgtggcagctatttctatatgtgttgctcgttggtgtgctgtgttcccattgtcgcaatttgttaactggatatatagagtaaagacaatcagatctatgagcggcataaccggagaaaatatttctgttcccgatgaaataccctacaattaccaaatgatgacattttgggcaggtttacgtggtgctgttggtgtcgccttggcgttgggaattcaaggtgagtataagttcactttattggcaacggtccttgttgttgttgttttaacagttatcatttttgggggcactactgcaggaatgttagaagttttaaatattaagactggttgcataagtgaagaagatacatctgatgacgagtttgatatagaggctccaagggcgataaatttattgaacggtagttctattcagacagatttgggcccatattctgacaacaattctccagatatttcaattgaccaattcgcggtcagcagtaacaagaatctccccaataacatatccacaactggtggtaatacttttggaggccttaatgaaactgagaatactagccctaacccggcaaggtcttctatggataagcgtaatttgagagataaactgggaacaatctttaattccgactcacaatggtttcaaaattttgatgaacaggtattgaagccagtattcttggacaacgtttctccatccttacaagattcggctacgcaatcacctgcagatttctcttcccaaaaccactag

SEQ ID NO: 24 - protein, 633 aa, Saccharomyces cerevisiae
MLSKVLLNIAFKVLLTTAKRAVDPDDDDELLPSPDLPGSDDPIAGDPDVDLNPVTEEMFSSWALFIMLLLLISALWSSYYLTQKRIRAVHETVLSIFYGMVIGLIIRMSPGHYIQDTVTFNSSYFFNVLLPPIILNSGYELNQVNFFNNMLSILIFAIPGTFISAVVIGIILYIWTFLGLESIDISFADAMSVGATLSATDPVTILSIFNAYKVDPKLYTIIFGESLLNDAISIVMFETCQKFHGQPATFSSVFEGAGLFLMTFSVSLLIGVLIGILVALLLKHTHIRRYPQIESCLILLIAYESYFFSNGCHMSGIVSLLFCGITLKHYAYYNMSRRSQITIKYIFQLLARLSENFIFIYLGLELFTEVELVYKPLLIIVAAISICVARWCAVFPLSQFVNWIYRVKTIRSMSGITGENISVPDEIPYNYQMMTFWAGLRGAVGVALALGIQGEYKFTLLATVLVVVVLTVIIFGGTTAGMLEVLNIKTGCISEEDTSDDEFDIEAPRAINLLNGSSIQTDLGPYSDNNSPDISIDQFAVSSNKNLPNNISTTGGNTFGGLNETENTSPNPARSSMDKRNLRDKLGTIFNSDSQWFQNFDEQVLKPVFLDNVSPSLQDSATQSPADFSSQNH

SEQ ID NO: 25 - DNA, 1857 bp, Magnaporthe grisea
atgactttcgatatcgccggcaacctcctggagctcaccaggcgcgctgccgaggaacccgaacctggaggaatggcagttggccttgccctgcgagtgtttgccgtcgatggactccaggacctcgtcagcttcgattaccaaatcttcttcaacctcctccttccacccatcatcctctcgtccggctacgagttacatcaggccaacttcttccggcacatcggaacaattctcacgttcgcatttgctggcacgttcctgtctgcagtagtcatcggtgttatactatggctttacactcgcgtacccctcgaggggctcaccatgaactggatcgatgccatatctgttggcgcaactttgtcagctaccgatcctgtcaccatcatagccatcttcaactcgtacaaggtggacccgaagctgtataccatcatctttggagaggccatcctcaatgacgctgtggccattgtcatcttcgagtcggcgcaaaagtccgccaggggcttgaccaaaggcagcgctgctggcatctctaccttcttctggggtttctggattttcttgagggacttcttcggcagcttgttcatcggggcgcttcttggcatcctcaccgcgctcatgctcaagtacacgtacctcaggaggtttcccaagctggagagctgcttgattgtgcttattgcttacgccacgtactacttttcccaggccatacacatgtctggaattgtgtcactgttgttctgcggaatcacactcaaacactatgcatacttcaacatgtcgcgaagaactcagcttacgaccaagtacatgttccaggtcctcgcacaactgtctgagaactttatctttatttacctgggtgtttccctctttacggacaaggatctccagttccagcccctcctcatcattgtcactgtcatggcggtgtgcgcagctcgctgggttgccgtattcccactctcgtgggccatcaactggttccacaagtaccgggcagaaagacgtggcatcaagaacgtgcccgaggagctgccgtacaagtaccaaggcatgctgttctgggcagggttgcgtggagcggtcggtgttgccctggccgcgttgttgacggccaaggaccaccgtgcattcaaggcgaccgttctggttgtggtggtgctcactgtcatcatatttggtggcactacggtcaacgtgcttgaaatcctcgagatccgcacgggagtgacggatgagatcgattctgacgatgaattcgacatcgaggcagttgggggctactacaagcgatcgggtaacggaataggttatagcccggccgggcgcaatggtgttgtgcccctggacacacgtccaggtcggagacgtgacagtaatggcgccgtcggtggaagagacgcgagcggctggagctcaggacatagatctcccttgagtgcggcaaggcctggcagtctcgtccgtacagggtcaacacgcgaagaagcggaaagactggacctccttggcaacccgggcggctcgacagactcggatgactttgggagcgacattgacacgtcggacctgccgccaccagcccctaggagacgatccagcccaatgccgcctacgggcgacgaagaggcagctggtttgccagcgggggggagcaggacaaggtcgaacacagagacgggtggcttgtcggccacggccgcgatccgccagctgttcagcaccgaggacccaacagccctgttcaggcagctggacgaggactacatcaaaccgaagctactgctcgatggcggtgccggccgtgggaacggtggtggcgctggcggatcgagttag

SEQ ID NO: 26 - protein, 618 aa, Magnaporthe grisea
MTFDIAGNLLELTRRAAEEPEPGGMAVGLALRVFAVDGLQDLVSFDYQIFFNLLLPPIILSSGYELHQANFFRHIGTILTFAFAGTFLSAVVIGVILWLYTRVPLEGLTMNWIDAISVGATLSATDPVTIIAIFNSYKVDPKLYTIIFGEAILNDAVAIVIFESAQKSARGLTKGSAAGISTFFWGFWIFLRDFFGSLFIGALLGILTALMLKYTYLRRFPKLESCLIVLIAYATYYFSQAIHMSGIVSLLFCGITLKHYAYFNMSRRTQLTTKYMFQVLAQLSENFIFIYLGVSLFTDKDLQFQPLLIIVTVMAVCAARWVAVFPLSWAINWFHKYRAERRGIKNVPEELPYKYQGMLFWAGLRGAVGVALAALLTAKDHRAFKATVLVVVVLTVIIFGGTTVNVLEILEIRTGVTDEIDSDDEFDIEAVGGYYKRSGNGIGYSPAGRNGVVPLDTRPGRRRDSNGAVGGRDASGWSSGHRSPLSAARPGSLVRTGSTREEAERLDLLGNPGGSTDSDDFGSDIDTSDLPPPAPRRRSSPMPPTGDEEAAGLPAGGSRTRSNTETGGLSATAAIRQLFSTEDPTALFRQLDEDYIKPKLLLDGGAGRGNGGGAGGSS

SEQ ID NO: 27 - DNA, 654 bp, Oryza sativa
cttctacatcggcttaggtgtagcaacacgactttattattattattattattattattattattttacaaaaatataaaatagatcagtccctcaccacaagtagagcaagttggtgagttattgtaaagttctacaaagctaatttaaaagttattgcattaacttatttcatattacaaacaagagtgtcaatggaacaatgaaaaccatatgacatactataattttgtttttattattgaaattatataattcaaagagaataaatccacatagccgtaaagttctacatgtggtgcattaccaaaatatatatagcttacaaaacatgacaagcttagtttgaaaaattgcaatccttatcacattgacacataaagtgagtgatgagtcataatattattttctttgctacccatcatgtatatatgatagccacaaagttactttgatgatgatatcaaagaacatttttaggtgcacctaacagaatatccaaataatatgactcacttagatcataatagagcatcaagtaaaactaacactctaaagcaaccgatgggaaagcatctataaatagacaagcacaatgaaaatcctcatcatccttcaccacaattcaaatattatagttgaagcatagtagta

SEQ ID NO: 28 - DNA, 941 bp, Oryza sativa
gataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttc

SEQ ID NO: 29 - DNA, 50 bp, Artificial sequence
  primer prm3122
ggggacaagtttgtacaaaaaagcaggcttcacaatggggatggaggtgg

SEQ ID NO: 30 - DNA, 48 bp, Artificial sequence
  primer prm3123
ggggaccactttgtacaagaaagctgggtgcactgttcatcttcctcc